(19) United States
(12) Patent Application Publication
(10) Pub. No.: US 2004/0157278 A1
(43) Pub. Date: Aug. 12, 2004

(54) Detection methods using TIMP 1

(75) Inventors: Jon H. Astle, Taunton, MA (US); Christopher C. Burgess, Westwood, MA (US); Theodore J. Catino, Attleboro, MA (US); Poornima Dwivedi, Alamo, CA (US); Maryanne Huntress, Attleboro, MA (US); Karen Anne Johnson, Acton, MA (US); Marcia E. Lewis, Cohasset, MA (US); Peter J. Maimonis, Westwood, MA (US); Gary A. Molino, Norfolk, MA (US); Susan H. Myerow, Lexington, MA (US); Arunthathi Thiagalingam, Lexington, MA (US); Lisa Allyn Boardman, Rochester, MN (US); Lawrence J. Burgart, Rochester, MN (US); Stephen N. Thibodeau, Rochester, MN (US); Sheryl Brown-Shimer, Boston, MA (US)

Correspondence Address: PALMER & DODGE, LLP, PAULA CAMPBELL EVANS, 111 HUNTINGTON AVENUE, BOSTON, MA, 02199, US

(73) Assignees: Bayer Corporation; Mayo Foundation for Medical Education & Research

(21) Appl. No.: 10/734,564

(22) Filed: Dec. 12, 2003

Related U.S. Application Data
(60) Provisional application No. 60/491,397, filed on Jul. 31, 2003.
(60) Provisional application No. 60/433,554, filed on Dec. 13, 2002.

(51) Int. Cl. G01N 33/567; G01N 33/574

(52) U.S. Cl. 435/7.23

(57) ABSTRACT

The present invention relates to a method for detecting the presence of colorectal cancer in an individual, wherein colorectal cancer is detected by detecting the presence of Reg1α or TIMP1 nucleic acid or amino acid molecules in a clinical sample obtained from the patient, wherein Reg1α or TIMP1 expression is indicative of the presence of colorectal cancer. The invention further relates to a method for detecting the presence of colorectal cancer in an individual, wherein colorectal cancer is detected by detecting the presence of Reg1α or TIMP1 nucleic acid or amino acid molecules in a clinical sample, in addition to detecting the presence of one or more additional colorectal cancer associated markers.

### RELATED APPLICATIONS

The present application claims priority to U.S. Patent Application Serial No. 60/433,554, filed Dec. 13, 2002 and U.S. Patent Application Serial No. 60/491,397, filed Jul. 13, 2003.

### BACKGROUND OF THE INVENTION

Colorectal carcinoma is a malignant neoplastic disease. There is a high incidence of colorectal carcinoma in the Western world, particularly in the United States. Tumors of this type often metastasize through lymphatic and vascular channels. Many patients with colorectal carcinoma eventually die from this disease. In fact, it is estimated that 62,000 persons in the United States alone die of colorectal carcinoma annually.

However, if diagnosed early, colorectal cancer may be treated effectively by surgical removal of the cancerous tissue. Colorectal cancers originate in the colorectal epithelium and typically are not extensively vascularized (and therefore not invasive) during the early stages of development. Colorectal cancer is thought to result from the clonal expansion of a single mutant cell in the epithelial lining of the colon or rectum. The transition to a highly vascularized, invasive and ultimately metastatic cancer which spreads throughout the body commonly takes ten years or longer. If the cancer is detected prior to invasion, surgical removal of the cancerous tissue is an effective cure. However, colorectal cancer is often detected only upon manifestation of clinical symptoms, such as pain and black tarry stool. Generally, such symptoms are present only when the disease is well established, often after metastasis has occurred, and the prognosis for the patient is poor, even after surgical resection of the cancerous tissue. Early detection of colorectal cancer therefore is important in that detection may significantly reduce its morbidity.

Invasive diagnostic methods such as endoscopic examination allow for direct visual identification, removal, and biopsy of potentially cancerous growths such as polyps. Endoscopy is expensive, uncomfortable, inherently risky, and therefore not a practical tool for screening populations to identify those with colorectal cancer. Non-invasive analysis of stool samples for characteristics indicative of the presence of colorectal cancer or precancer is a preferred alternative for early diagnosis, but no known diagnostic method is available which reliably achieves this goal. A reliable, non-invasive, and accurate technique for diagnosing colorectal cancer at an early stage would help save many lives.

Ectopic expression of the pancreatic regenerating gene (RegI) has been identified previously in colorectal tumors, and suggested as a potential marker for colorectal cancer (Zenilman et al., (1997) J. Gastrointest. Surg., 1: 194; Watanabe et al., (1990) J. Biol. Chem., 265: 7432; Birse and Rosen, WO01/12781). At present, there is no reliable method known to those of skill in the art for the rapid and accurate detection of Reg1α in the serum of colorectal cancer patients (Satomura et al., (1995) J. Gastroenterol. 30: 643). There is thus a need in the art for a method of detecting, and/or monitoring colorectal cancer in a patient utilizing the expression of Reg1α in serum.

### SUMMARY OF THE INVENTION

The present invention is provides a method of detecting, monitoring or determining the therapeutic response of colorectal cancer in an individual as well as compositions, and kits for performing the method. In its most general aspect, the method comprises: obtaining a clinical sample from the individual and detecting the presence of Reg1α or TIMP1 in said sample, wherein the presence of Reg1α or TIMP1 in the sample is indicative of the presence or stage of colorectal cancer in the individual.

In one embodiment, the step of detecting comprises: contacting said clinical sample with a ligand which is capable of binding to Reg1α or TIMP1 under conditions which permit the ligand to bind to Reg1α or TIMP1; and detecting the binding of the ligand to Reg1α or TIMP1, wherein detection of binding is indicative of the presence of Reg1α or TIMP1 in the sample. The polypeptide ligand may comprise, for example, an antibody, peptide, oligonucleotide, or other molecule that specifically binds Reg1α or TIMP1. In a currently preferred embodiment, the clinical sample comprises serum.

The present invention further provides a method of detecting, monitoring, or determining the presence of colorectal cancer in an individual comprising: obtaining a clinical sample from said individual; and detecting the presence of Reg1α or TIMP1 and at least one other colorectal cancer associated marker in the sample, wherein the presence of Reg1α or TIMP1 and the at least one other colorectal cancer associated marker is indicative of colorectal cancer in the individual. The colorectal cancer associated marker may comprise, for example, one or more of the nucleic acid sequences of SEQ ID Nos 1, 3, or 5-71, or the amino acid sequences of SEQ ID Nos 2, 4, or 72-138, or derivatives or homologs thereof having substantially the same binding specificity.

In a preferred embodiment embodiment, the above step of detecting comprises contacting a serum sample with a first ligand which is capable of binding to Reg1α or TIMP1 and a second ligand which is capable of binding to the colorectal cancer associated marker, under conditions which permit the first and second ligands to bind to Reg1α or TIMP1 and the colorectal cancer associated marker, respectively; and detecting the binding of the first ligand to Reg1α or TIMP1 and the second ligand to the colorectal cancer associated marker, wherein detection of binding is indicative of the presence of Reg1α or TIMP1 and the colorectal cancer associated marker in said sample. The polypeptide ligand may comprise, for example, an antibody, peptide, oligonucleotide, or other molecule that specifically binds Reg1α or TIMP1.

The present invention also provides a method of detecting, monitoring or determining the presence of colorectal cancer in an individual comprising: obtaining a clinical sample from an individual; and detecting the presence of a nucleic acid molecule which encodes Reg1α or TIMP1 in said sample, wherein the presence of the nucleic acid molecule in the sample is indicative of colorectal cancer in the individual.

The invention still further provides a method of detecting, monitoring or determining the presence of colorectal cancer in an individual comprising: obtaining a clinical sample from an individual; and detecting the presence of a nucleic acid molecule which encodes Reg1α or TIMP1 and at least one other nucleic acid molecule which encodes at least one other colorectal cancer associated marker in the sample, wherein the presence of the nucleic acid sequence encoding Reg1α or TIMP1 and the nucleic acid sequence encoding the at least one other colorectal cancer associated marker is indicative of colorectal cancer in the individual. In a preferred embodiment, the colorectal cancer associated marker is one or more of the nucleic acid sequences of SEQ ID Nos 1, 3, or 5-71, or the amino acid sequences of SEQ ID Nos 2, 4, or 72-138, or derivatives or homologs thereof having substantially the same binding specificity.

### BRIEF DESCRIPTION OF THE DRAWINGS

FIG. 1 shows the level of Reg1α polypeptide present in serum obtained from normal control patients (n=35), patients diagnosed with inflammatory bowel disease (IBD; n=7), patients diagnosed with cirrhosis (n=7), and patients diagnosed with colorectal cancer (n=63).

FIG. 2 shows the level of Reg1α polypeptide measured in the colorectal cancer patient group (n=63) differentiated based on cancer severity. The degree of cancer has been established by Dukes'-type staging, and data from patients with Dukes'-type A, B, C, and D is shown.

FIG. 3 shows a graphical representation of the plasma level of TIMP1 polypeptides, along with one or more other colorectal cancer associated markers obtained from patients with colorectal cancer.

### DETAILED DESCRIPTION

The present invention is based, in part, on the discovery that the expression of they human islet regenerating protein, Reg1α, is increased in patients with colorectal cancer, and as such is a valuable marker for the identification of colorectal cancer in humans. The present invention further provides for the early detection of colorectal cancer by detecting the presence of Reg1α or TIMP1 (and optionally, one or more additional colorectal cancer associated markers) in a clinical sample from an individual. The invention provides further, the ability to monitor the recurrence of colorectal cancer in a patient wherein colorectal cancer has been previously detected, by monitoring the levels of Reg1α or TIMP1 polypeptide or polynucleotide sequences present in a clinical sample from the patient, wherein an increase in Reg1α or TIMP1 in the sample is indicative of the recurrence of colorectal cancer. The invention provides still further, the ability to monitor the decrease in colorectal cancer in response to a therapeutic agent, whereby the levels of Reg1α or TIMP1 are measured in a clinical sample obtained from a patient who has received therapeutic treatment for colorectal cancer, wherein a decrease in the levels of Reg1α or TIMP1 detected in the clinical sample from the patient is indicative of the efficacy of the therapeutic treatment. In any of the preceding embodiments, Reg1α or TIMP1 polynucleotide or polypeptide expression levels are measured in concert with at least one additional colorectal cancer associated marker.

Accordingly, the present invention relates in part to novel methods for identifying cancer in an individual, particularly colorectal cancer, by screening for genes or gene products, which are over or underexpressed in cancer relative to the level of expression in normal tissue, such as colon tissue. Alternatively, the invention provides a method for the identification of cancer in an individual by screening for genes or gene products which are over- or underexpressed in colorectal cancer, and which are detectable in a clinical sample of an individual with colorectal cancer.

In a preferred embodiment, the present invention relates to methods useful for the detection of colorectal cancer in an individual, preferably a human patient by detecting serum levels of Reg1α or TIMP1. The invention relates to methods for colorectal cancer detection that utilize either or both techniques of detecting the presence of the Reg1α or TIMP1 gene or detecting the Reg1α or TIMP1 encoded polypeptide product in the serum of an individual, or alternatively in a clinical sample from an individual.

The present invention further provides methods for the identification of colorectal cancer wherein cancer is detected by the identification of Reg1α or TIMP1 expression in a patient clinical sample, in combination with the expression in the same sample of at least one other colorectal cancer associated marker. This combination of Reg1α or TIMP1 detection analysis, in concert with the detection of additional colon-cancer markers provides an efficient and reliable method for detecting the presence of colorectal cancer.

The methods described herein which specifically refer to the detection of Reg1α, may equally be applied to the detection of TIMP1 by one of skill in the art, based on the disclosure of the present specification.

Definitions

As used herein, “Reg1α” refers to a polypeptide molecule having the sequence of either of SEQ ID Nos 2 or 4. Reg1α as used herein, also refers to a polypeptide which is encoded by either of the sequences of SEQ ID Nos. 1 or 3. The sequences of SEQ ID Nos 2 and 4 each represent a functional Reg1α protein, but differ from each other by four amino acids in the leader sequence which is cleaved off during protein synthesis.

As used herein, “TIMP1” refers to a polypeptide molecule having the sequence of SEQ ID NO: 100. TIMP1 as used herein, also refers to a nucleotide which is encoded by the sequence of SEQ ID NO: 33, or a functional homolog thereof.

As used herein, a “clinical sample” refers to a tissue, cellular, or fluid sample obtained from an individual. A “clinical sample”, as used herein, can refer to a cells, circulating cells (e.g., circulating cells in blood), cells obtained from specific anatomical locations, or specific cell types (e.g., colon cell, gastrointestinal cell, cancerous cell, etc.), a tissue sample, or physiological fluids such as lymph, bile, serum, plasma, urine, synovial fluid, blood, CSF, mucus membrane secretions, or other physiological samples such as stool. Preferably, the clinical sample is serum or plasma. A colorectal cancer associated marker of the invention, such as TIMP1, may be detected in a suitable “clinical sample” where the suitability of a particular type of clinical sample for the detection of a specific colorectal cancer associated marker may be readily determined by one of skill in the art.

As used herein, “detecting” refers to the identification of the presence or absence of a molecule in a sample. Where the molecule to be detected is a polypeptide, the step of detecting can be performed by binding the polypeptide with an antibody that is detectably labeled. A detectable label is a molecule which is capable of generating, either independently, or in response to a stimulus, an observable signal. A detectable label can be, but is not limited to a fluorescent label, a chromogenic label, a luminescent label, or a radioactive label. Methods for “detecting” a label include quantitative and qualitative methods adapted for standard or confocal microscopy, FACS analysis, and those adapted for high throughput methods involving multiwell plates, arrays or microarrays. One of skill in the art can select appropriate filter sets and excitation energy sources for the detection of fluorescent emission from a given fluorescent polypeptide or dye. “Detecting” as used herein can also include the use of multiple antibodies to a polypeptide to be detected, wherein the multiple antibodies bind to different epitopes on the polypeptide to be detected. Antibodies used in this manner can employ two or more detectable labels, and can include, for example a FRET pair. A polypeptide molecule, such as Reg1α, is “detected” according to the present invention when the level of detectable signal is at all greater than the background level of the detectable label, or where the level of measured nucleic acid is at all greater than the level measured in a control sample.

As used herein, “detecting” as it refers to detecting the presence of a target nucleic acid molecule (e.g., a nucleic acid molecule encoding Reg1α, or other colorectal cancer-specific sequence) refers to a process wherein the signal generated by a directly or indirectly labeled probe nucleic acid molecule (capable of hybridizing to a target, e.g., a sequence encoding Reg1α, in a serum sample) is measured or observed. Thus, detection of the probe nucleic acid is directly indicative of the presence, and thus the detection, of a target nucleic acid, such as a sequence encoding Reg1α. For example, if the detectable label is a fluorescent label, the target nucleic acid (e.g., the nucleic acid molecule encoding Reg1α) is “detected” by observing or measuring the light emitted by the fluorescent label on the probe nucleic acid when it is excited by the appropriate wavelength, or if the detectable label is a fluorescence/quencher pair, the target nucleic acid is “detected” by observing or measuring the light emitted upon association or dissociation of the fluorescence/quencher pair present on the probe nucleic acid, wherein detection of the probe nucleic acid indicates detection of the target nucleic acid. If the detectable label is a radioactive label, the target nucleic acid, following hybridization with a radioactively labeled probe is “detected” by, for example, autoradiography. Methods and techniques for “detecting” fluorescent, radioactive, and other chemical labels may be found in Ausubel et al. (1995, Short Protocols in Molecular Biology, 3^{rd} Ed. John Wiley and Sons, Inc.). Alternatively, a nucleic acid may be “indirectly detected” wherein a moiety is attached to a probe nucleic acid which will hybridize with the target, such as an enzyme activity, allowing detection in the presence of an appropriate substrate, or a specific antigen or other marker allowing detection by addition of an antibody or other specific indicator. Alternatively, a target nucleic acid molecule can be detected by amplifying a nucleic acid sample prepared from a patient clinical sample, using oligonucleotide primers which are specifically designed to hybridize with a portion of the target nucleic acid sequence. Quantative amplification methods, such as, but not limited to TaqMan, may also be used to “detect” a target nucleic acid according to the invention. A nucleic acid molecule is “detected” as used herein where the level of nucleic acid measured (such as by quantitative PCR), or the level of detectable signal provided by the detectable label is at all above the background level.

As used herein, “detecting” refers further to the early detection of colorectal cancer in a patient, wherein “early” detection refers to the detection of colorectal cancer at Dukes stage A or preferably, prior to a time when the colorectal cancer is morphologically able to be classified in a particular Dukes stage. “Detecting” as used herein further refers to the detection of colorectal cancer recurrence in an individual, using the same detection criteria as indicated above. “Detecting” as used herein still further refers to the measuring of a change in the degree of colorectal cancer before and/or after treatment with a therapeutic agent. In this case, a change in the degree of colorectal cancer in response to a therapeutic agent refers to an increase or decrease in the expression of Reg1α (and optionally, one or more additional colorectal cancer associated markers), or alternatively, in the amount of Reg1α polypeptide (and optionally, one or more additional colorectal cancer associated markers) present in a clinical sample by at least 10% in response to the presence of a therapeutic agent relative to the expression level in the absence of the therapeutic agent.

As used herein, “individual” refers to a mammal, preferably a human.

As used herein, a “ligand” refers to a molecule which is capable of binding a polypeptide. A “polypeptide ligand” useful in the present invention includes, but is not limited to an antibody, a monoclonal antibody, a polyclonal antibody, an antibody fragment (e.g., Fv, scFV, or Fab), a small molecule, or a nucleic acid aptamer. A “ligand” as used herein can also refer to a “nucleic acid ligand”, such as an oligonucleotide, polynucleotide, DNA, RNA, mRNA, or cDNA, which is capable of binding to a complementary nucleic acid molecule, or polypeptide molecule.

The term “antibody” as used herein is intended to include whole antibodies, e.g., of any isotype (IgG, IgA, IgM, IgE, etc), and includes fragments thereof, and single-chain antibodies, which also are specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)₂, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

As used herein, a “colorectal cancer associated marker” refers to a polypeptide or nucleic acid sequence which exhibits over- or underexpression of at least 10% in colorectal cancer cells, tissue, or serum obtained from an individual having colorectal cancer, relative to the level of expression in cells, tissue, or serum obtained from an individual that does not have colorectal cancer. Non-limiting examples of colorectal cancer associated markers useful in the present invention include the nucleic acid molecules of SEQ ID Nos 1, 3, 5-71, and/or the polypeptide molecules of SEQ ID Nos 2, 4, 72-138. In one embodiment, the polypeptide sequences of SEQ ID Nos 2, 4, 72-138 are encoded by the nucleic acid sequences of 1, 3, 5-71, respectively. A “colorectal cancer specific marker” useful in the invention may be a polypeptide or nucleic acid sequence which exhibits over- or underexpression in colorectal cancer as described above, but which may also be over or underexpressed in other, non-colorectal types of cancer. Alternatively, a “colorectal cancer associated marker”, as used herein, may refer to a carbohydrate epitope present on a polypeptide or nucleic acid molecule and/or an antibody molecule which recognizes and is capable of binding to such an epitope, wherein the carbohydrate epitope is known to be associated with the presence of colorectal cancer in an individual. Such carbohydrate epitopes may be present on more than one unrelated protein or polypeptide. In one embodiment, such a carbohydrate epitope is CA 19-9, also known as sialyl-Lewis^{a}, is a tumor marker defined by a monoclonal antibody as a carbohydrate epitope, related to the blood group antigens, composed of a branching, 5-sugar structure covalently bound to a variety of glycoproteins or glycolipids. The proteins primarily belong to the mucin family and the lipids are usually membrane associated. The CA 19-9 epitope is typically the terminal moiety of a complex, O-linked carbohydrate structure on either macromolecule. Other tumor markers also defined as various carbohydrate epitopes useful in the present invention as a “colorectal cancer associated marker” include CA 72-4, TF, sTn, Tn, CA 50, CA 549, CA 242, LASA, and the Du-PAN's 1-5.

The term “interact” as used herein is meant to include detectable interactions (e.g., biochemical interactions) between molecules, such as interaction between protein-protein, protein-nucleic acid, nucleic acid-nucleic acid, and protein-small molecule or nucleic acid-small molecule in nature.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

The terms “protein”, “polypeptide”, and “peptide” are used interchangeably herein when referring to a gene product. As used herein, “polypeptide” refers to any kind of polypeptide such as peptides, human proteins, fragments of human proteins, proteins or fragments of proteins from non-human sources, engineered versions proteins or fragments of proteins, enzymes, antigens, drugs, molecules involved in cell signaling, such as receptor molecules, antibodies, including polypeptides of the immunoglobulin superfamily, such as antibody polypeptides or T-cell receptor polypeptides.

As used herein, the term “level of expression” refers to the measurable expression level of a given nucleic acid. The level of expression of a nucleic acid is determined by methods well known in the art. The “level of expression” may measured by hybridization analysis using labeled target nucleic acids according to methods well known in the art (see, for example, Ausubel et al., Short Protocols in Molecular Biology, 3^{rd} Ed. 1995, John Wiley and Sons, Inc.). The label on the target nucleic acid is a luminescent label, an enzymatic label, a radioactive label, a chemical label or a physical label. Preferably, the target nucleic acids are labeled with a fluorescent molecule. Preferred fluorescent labels include fluorescein, amino coumarin acetic acid, tetramethylrhodamine isothiocyanate (TRITC), Texas Red, Cy3 and Cy5. Alternatively, the “level of expression” can be measured by quantitative amplification protocols, such as TaqMan, known to those of skill in the art.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

Reg1α and TIMP1 Nucleic Acid

As described above, the present invention relates to the detection of Reg1α or TIMP1 polypeptide in a clinical sample from an individual, preferably a serum or plasma sample, thus permitting the detection of colorectal cancer. The present invention, however, equally relates to the identification of the nucleic acid sequence which encodes Reg1α or TIMP1 as a marker for colorectal cancer.

Nucleic acid and amino acid sequences of Reg1α are shown in SEQ ID Nos 1 or 3, and 2 or 4, respectively. Nucleic acid and amino acid sequences of TIMP1 are shown in SEQ ID NO: 33 and 100 respectively. While the invention relates to the direct detection of either of the sequences of Reg1α or TIMP1 in a method for detecting colorectal cancer, the invention further relates to the detection of sequences complementary thereto, or a sequence which specifically hybridizes to a sequence of SEQ ID Nos. 1, 3, or 33. The present invention also relates to the detection of colorectal cancer by detecting the presence, in a clinical sample, of a nucleic acid molecule which encodes the sequence of SEQ ID Nos. 2, 4, or 100, or a fragment thereof.

Another aspect of the invention provides the detection of colorectal cancer by the detection of a nucleic acid which hybridizes under low, medium, or high stringency conditions to a nucleic acid sequence represented by one or more of SEQ ID Nos. 1, 3, or 33, or a sequence complementary thereto. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-12.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In a preferred embodiment, a nucleic acid encoding Reg1α or TIMP1 will bind to SEQ ID Nos. 1, 3 or 33, or a sequence complementary thereto, or a fragment thereof, under moderately stringent conditions, for example at about 2.0×SSC and about 40° C. In a particularly preferred embodiment, a Reg1α or TIMP1 nucleic acid sequence present in a patient clinical sample will bind of SEQ ID Nos. 1, 3, or 33, respectively, or a sequence complementary thereto, or fragment thereof, under high stringency conditions.

In one embodiment, the invention provides nucleic acids which hybridize under low stringency conditions of 6×SSC at room temperature followed by a wash at 2×SSC at room temperature.

In another embodiment, the invention provides nucleic acids which hybridize under high stringency conditions of 2×SSC at about 65° C. followed by a wash at 0.2×SSC at about 65° C.

Detection of Reg1α nucleic acids having a sequence that differs from the nucleotide sequences shown in SEQ ID Nos. 1 or 3, or a sequence complementary thereto, due to degeneracy in the genetic code, are also within the scope of the invention. Such nucleic acids encode functionally equivalent peptides (i.e., a peptide having equivalent or similar biological activity) but differ in sequence from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a polypeptide. However, it is expected that DNA sequence polymorphisms that do lead to changes in the amino acid sequences of the subject polypeptides will exist among mammals. One skilled in the art will appreciate that these variations in one or more nucleotides (e.g., up to about 3-5% of the nucleotides) of the nucleic acids encoding polypeptides having an activity of a polypeptide may exist among individuals of a given species due to natural allelic variation.

The invention also includes within its scope a polynucleotide which hybridizes under stringent conditions (at least about 4×SSC at 65° C., or at least about 4×SSC at 42° C.; see, for example, U.S. Pat. No. 5,707,829, incorporated herein by reference) with at least 15 contiguous nucleotides of SEQ ID Nos. 1 or 3. By this is intended that when at least 15 contiguous nucleotides of SEQ ID Nos. 1 or 3 is used as a probe, the probe will preferentially hybridize with a gene or mRNA (of the biological material) comprising the complementary sequence, allowing the identification and retrieval of the nucleic acids (i.e., Reg1α) of the biological material that uniquely hybridize to the selected probe. Probes of more than 15 nucleotides can be used, but 15 nucleotides represents enough sequence for unique identification.

Constructs of polynucleotides having the sequence of SEQ ID Nos. 1 or 3, a portion thereof, or a sequence complementary thereto, and useful, for example for generating a probe, can be produced synthetically, or obtained from natural sources (e.g., human cells) using methods well known to those of skill in the art (see, for example, Sambrook et al., Molecular Cloning: A Laboratory Manual, 2nd Ed. (Cold Spring Harbor Press, Cold Spring Harbor, N.Y. 1989).

Calculation of Sequence Homology

In one embodiment, the present invention relates to the detection of colorectal cancer in an individual by detecting the presence of Reg1α or TIMP1 or a sequence homologous thereto, by using probes and/or primers which are complementary to portions of the Reg1α a or TIMP1 sequence, or are sufficiently homologous to portions of the Reg1α or TIMP1 sequence to permit hybridization of the probes and/or primers to Reg1α or TIMP1 under high stringency conditions. Sequences of the invention are at least 50% homologous to Reg1α or TIMP1, and are preferably 60%, 70%, 80%, 90% homologous up to complete sequence identity with Reg1α or TIMP1 (or optionally to a sequence encoding one or more additional colorectal cancer associated markers).

Sequence identity with respect to any of the sequences presented herein can be determined by a simple “eyeball” comparison (i.e. a strict comparison) of any one or more of the sequences with another sequence to see if that other sequence has, for example, at least 80% sequence identity to the sequence(s).

Relative sequence identity can also be determined by commercially available computer programs that can calculate % identity between two or more sequences using any suitable algorithm for determining identity, using for example default parameters. A typical example of such a computer program is CLUSTAL. Other computer program methods to determine identity and similarity between two sequences include but are not limited to the GCG program package (Devereux et al 1984 Nucleic Acids Research 12: 387) and FASTA (Atschul et al 1990 J Molec Biol 403-410).

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (Ausubel et al., 1995, Short Protocols in Molecular Biology, 3rd Edition, John Wiley & Sons), FASTA (Atschul et al., 1990, J. Mol. Biol., 403-410) and the GENEWORKS suite of comparison tools. Both BLAST and FASTA are available for offline and online searching (Ausubel et al., 1999 supra, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail on the World Wide Web at ncbi.nih.gov/BLAST/blast_help.html, which is incorporated herein by reference. The search parameters are defined as follows, and can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available on the World Wide Web at ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided on the World Wide Web at ncbi.nlm.nih.gov/BLAST. In some embodiments of the present invention, no gap penalties are used when determining sequence identity.

Probes and Primers

The nucleotide sequence of Reg1α or TIMP1 is useful in the present invention for the generation of probes and primers designed for identifying the Reg1α or TIMP1 nucleic acid sequence in a patient sample such as serum, colon cells or tissue. Nucleotide sequences useful as probes/primers may include all or a portion of SEQ ID Nos. 1, 3 or 33, or a sequence complementary thereto, or sequences which hybridize under stringent conditions to all or a portion of SEQ ID No. 1, 3 or 33. For instance, the present invention also provides a probe/primer comprising a substantially purified oligonucleotide, which oligonucleotide comprising a nucleotide sequence that hybridizes under stringent conditions to at least approximately 8, preferably about 12, preferably about 15, preferably about 25, more preferably about 40 consecutive nucleotides up to the full length of the sense or anti-sense sequence of SEQ ID Nos. 1, 3 or 33, or a sequence complementary thereto, or a naturally occurring mutant thereof. For instance, primers based on the nucleic acid represented in SEQ ID No. 1, 3 or 33, or a sequence complementary thereto, can be used in a reaction to amplify a template nucleic acid (e.g., Reg1α) contained within an mRNA sample derived from a patient clinical sample.

Not only are probes based on the nucleic acid sequence encoding Reg1α or TIMP1 useful for detecting Reg1α or TIMP1, but they can also provide a method for detecting mutations in wild-type Reg1α or TIMP1 in a patient. Nucleic acid probes which are complementary to a wild-type Reg1α or TIMP1 and can form mismatches with mutant genes are provided, allowing for detection by enzymatic or chemical cleavage or by shifts in electrophoretic mobility. Likewise, probes based on the subject sequences can be used to detect transcripts or genomic sequences encoding the same or homologous proteins, for use, for example, in prognostic or diagnostic assays. In preferred embodiments, the nucleic acid probe further comprises a label group attached thereto and able to be detected, e.g., the label group is selected from a radioisotope, a fluorescent compound, a chemiluminescent compound, a chromagenic compound, an enzyme, and enzyme co-factor.

Full-length cDNA molecules comprising the disclosed nucleic acids, useful for the generation of probes, primers, or for transcription to produce the Reg1α or TIMP1 protein itself, or antibodies thereto may be obtained as follows. The nucleic acid sequence of Reg1α or TIMP1 or a portion thereof comprising at least approximately 8, preferably about 12, preferably about 15, preferably about 25, more preferably about 40 nucleotides up to the full length of the sequence of SEQ ID Nos. 1, 3 or 33, or a sequence complementary thereto, may be used as a hybridization probe to detect hybridizing members of a cDNA library using probe design methods, cloning methods, and clone selection techniques as described in U.S. Pat. No. 5,654,173, “Secreted Proteins and Polynucleotides Encoding Them,” incorporated herein by reference. Libraries of cDNA may be made from selected tissues, such as normal or tumor tissue, or from tissues of a mammal treated with, for example, a pharmaceutical agent. Preferably, the tissue is the same as that used to generate the nucleic acids, as both the nucleic acid and the cDNA represent expressed genes. Alternatively, many cDNA libraries are available commercially. (Sambrook et al., Molecular Cloning: A Laboratory Manual, 2nd Ed. (Cold Spring Harbor Press, Cold Spring Harbor, N.Y. 1989). The choice of cell type for library construction may be made after the identity of the protein encoded by the nucleic acid-related gene is known. This will indicate which tissue and cell types are likely to express the related gene, thereby containing the mRNA for generating the cDNA.

Members of the library that are larger than the nucleic acid, and preferably that contain the whole sequence of the native message, may be obtained. To confirm that the entire cDNA has been obtained, RNA protection experiments may be performed as follows. Hybridization of a full-length cDNA to an mRNA may protect the RNA from RNase degradation. If the cDNA is not fill length, then the portions of the mRNA that arc not hybridized may be subject to RNase degradation. This may be assayed, as is known in the art, by changes in electrophoretic mobility on polyacrylamide gels, or by detection of released monoribonucleotides. Sambrook et al., Molecular Cloning: A Laboratory Manual, 2nd Ed. (Cold Spring Harbor Press, Cold Spring Harbor, N.Y. 1989). In order to obtain additional sequences 5′ to the end of a partial cDNA, 5′ RACE (PCR Protocols: A Guide to Methods and Applications (Academic Press, Inc. 1990)) may be performed.

Genomic DNA (e.g., Reg1α genomic DNA) may be isolated using nucleic acids in a manner similar to the isolation of full-length cDNAs. Briefly, the nucleic acids, or portions thereof, may be used as probes to libraries of genomic DNA. Preferably, the library is obtained from the cell type that was used to generate the nucleic acids. Most preferably, the genomic DNA is obtained from the biological material described herein in the Example. Such libraries may be in vectors suitable for carrying large segments of a genome, such as P1 or YAC, as described in detail in Sambrook et al., pages 9.4-9.30. In addition, genomic sequences can be isolated from human BAC libraries, which are commercially available from Research Genetics, Inc., Huntville, Ala., USA, for example. In order to obtain additional 5′ or 3′ sequences, chromosome walking may be performed, as described in Sambrook et al., such that adjacent and overlapping fragments of genomic DNA are isolated. These may be mapped and pieced together, as is known in the art, using restriction digestion enzymes and DNA ligase.

Using the nucleic acids of the invention, corresponding full length genes can be isolated using both classical and PCR methods to construct and probe cDNA libraries. Using either method, Northern blots, preferably, may be performed on a number of cell types to determine which cell lines express the gene of interest at the highest rate.

Classical methods of constructing cDNA libraries in Sambrook et al., supra. With these methods, cDNA can be produced from mRNA and inserted into viral or expression vectors. Typically, libraries of mRNA comprising poly(A) tails can be produced with poly(T) primers. Similarly, cDNA libraries can be produced using the instant Reg1α sequence or portions thereof as primers.

PCR methods may be used to amplify the members of a cDNA library that comprise the desired insert. In this case, the desired insert may contain sequence from the full length cDNA that corresponds to the sequence encoding Reg1α. Such PCR methods include gene trapping and RACE methods.

Gene trapping may entail inserting a member of a cDNA library into a vector. The vector then may be denatured to produce single stranded molecules. Next, a substrate-bound probe, such a biotinylated oligo, may be used to trap cDNA inserts of interest. Biotinylated probes can be linked to an avidin-bound solid substrate. PCR methods can be used to amplify the trapped cDNA. To trap sequences corresponding to the full length genes, the labeled probe sequence may be based on the nucleic acid of SEQ ID Nos. 1 or 3, or a sequence complementary thereto. Random primers or primers specific to the library vector can be used to amplify the trapped cDNA. Such gene trapping techniques are described in Gruber et al., PCT WO 95/04745 and Gruber et al., U.S. Pat. No. 5,500,356. Kits are commercially available to perform gene trapping experiments from, for example, Life Technologies, Gaithersburg, Md., USA.

“Rapid amplification of cDNA ends,” or RACE, is a PCR method of amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In preferred embodiments of RACE, a common primer may be designed to anneal to an arbitrary adaptor sequence ligated to cDNA ends (Apte and Siebert, Biotechniques 15:890-893, 1993; Edwards et al., Nuc. Acids Res. 19:5227-5232, 1991). When a single gene-specific RACE primer is paired with the common primer, preferential amplification of sequences between the single gene specific primer and the common primer occurs. Commercial cDNA pools modified for use in RACE are available.

Once the full-length cDNA or gene is obtained, DNA encoding variants can be prepared by site-directed mutagenesis, described in detail in Sambrook 15.3-15.63. The choice of codon or nucleotide to be replaced can be based on the disclosure herein on optional changes in amino acids to achieve altered protein structure and/or function.

As an alternative method to obtaining DNA or RNA from a biological material, such as serum, nucleic acid comprising nucleotides having the sequence of one or more nucleic acids of the invention can be synthesized. Thus, the invention encompasses nucleic acid molecules ranging in length from about 8 nucleotides (corresponding to at least 12 contiguous nucleotides which hybridize under stringent conditions to or are at least 80% identical to the nucleic acid sequence of SEQ ID Nos. 1 or 3, or a sequence complementary thereto) up to a maximum length suitable for one or more biological manipulations, including replication and expression, of the nucleic acid molecule. The invention includes but is not limited to (a) nucleic acid having the size of the full Reg1α gene, or a sequence complementary thereto; (b) the nucleic acid of (a) also comprising at least one additional gene, operably linked to permit expression of a fusion protein; (c) an expression vector comprising (a) or (b); (d) a plasmid comprising (a) or (b); and (e) a recombinant viral particle comprising (a) or (b).

The sequence of a nucleic acid of the present invention is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired.

Probe Preparation

Prior to hybridization of a probe nucleic acid to a patient sample, the nucleic acid samples must be prepared to facilitate subsequent detection of hybridization. The nucleic acid samples obtained from an individual (including nucleic acid sequences encoding Reg1α, and optionally, at least one other colorectal cancer associated marker) to be screened for colorectal cancer are capable of being bound by a nucleic acid probe of complementary sequence through one or more types of chemical bonds, usually through complementary base pairing, usually through hydrogen bond formation.

Probes useful in the invention for hybridizing to and thus identifying the presence of Reg1α or TIMP1, and optionally, at least one additional colorectal cancer associated marker may be designed to hybridize to a polynucleotide molecule derived from an mRNA transcript coding for Reg1α, or optionally, at least one additional colorectal cancer associated marker. As used herein, a “polynucleotide derived from an mRNA transcript” refers to a polynucleotide for which synthesis of the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, suitable target nucleic acid samples include, but are not limited to, mRNA transcripts of a gene or genes (i.e., Reg1α or a colorectal cancer associated marker), cDNA reverse transcribed from the mRNA, cRNA transcribed from the cDNA, DNA amplified from a gene or genes, RNA transcribed from amplified DNA, and the like. The polynucleotide probes used herein are preferably designed to hybridize to Reg1α, or optionally to a sequence encoding at least one other colorectal cancer associated marker.

Nucleic acid probes may be generated using techniques which are well known to those of skill in the art (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987).

In order to measure the hybridization of a probe nucleic acid to a target sequence in a sample, the probe nucleic acid is preferably labeled with a detectable label. Any analytically detectable marker that is attached to or incorporated into a molecule may be used in the invention. An analytically detectable marker refers to any molecule, moiety or atom which is analytically detected and quantified.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, 35S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The labels may be incorporated into a nucleic acid probe by any of a number of means well known to those of skill in the art. However, in a preferred embodiment, the label is simultaneously incorporated into the probe during an amplification step in the preparation of the probe polynucleotides. Thus, for example, polymerase chain reaction (PCR), or other amplification reaction, with labeled primers or labeled nucleotides will provide a labeled amplification product, and thus a labeled probe.

Alternatively, a label may be added directly to the probe. Means of attaching labels to polynucleotides are well known to those of skill in the art and include, for example nick translation or end-labeling (e.g. with a labeled RNA) and subsequent attachment (ligation) of a polynucleotide linker joining the sample polynucleotide to a label (e.g., a fluorophore).

In a preferred embodiment, the fluorescent modifications are by cyanine dyes e.g. Cy-3/Cy-5 dUTP, Cy-3/Cy-5 dCTP (Amersham Pharmacia) or alexa dyes (Khan, J., Simon, R., Bittner, M., Chen, Y., Leighton, S. B., Pohida, T., Smith, P. D., Jiang, Y., Gooden, G. C., Trent, J. M. & Meltzer, P. S. (1998) Cancer Res. 58, 50095013.).

In a preferred embodiment, a probe nucleic acid which is capable of hybridizing to Reg1α and a probe nucleic acid which is capable of hybridizing to a nucleic acid sequence encoding at least one additional colorectal cancer associated marker, are co-hybridized to a test sample (e.g., a serum sample). In this embodiment, the two probe samples used for comparison are labeled with different fluorescent dyes which produce distinguishable detection signals, for example, probes hybridizable with Reg1α are labeled with Cy5 and probes hybridizable with another colorectal cancer associated marker are labeled with Cy3. The differently labeled target samples are hybridized to the same microarray simultaneously.

In a preferred embodiment, a control probe may be co-hybridized to a sample along with a probe for Reg1α and/or a probe for an additional colorectal cancer associated marker, wherein the control probe is capable of hybridizing to a nucleic acid sequence known to be found in the clinical sample, for example, where the clinical sample is a serum sample, a control sequence may be a sequence encoding serum albumin, or fibrinogen.

Vectors and Host Cells

The present invention further provides vectors and plasmids useful for directing the expression of Reg1α or TIMP1 or other colorectal cancer associated markers, and further provides host cells which express the vectors and plasmids provided herein. Nucleic acid sequences useful for the expression from a vector or plasmid as described below include, but are not limited to any nucleic acid or gene sequence identified as being differentially regulated by the methods described above, and further include therapeutic nucleic acid molecules, such as antisense molecules. The host cell may be any prokaryotic or eukaryotic cell. Ligating the polynucleotide sequence into a gene construct, such as an expression vector, and transforming or transfecting into hosts, either eukaryotic (yeast, avian, insect or mammalian) or prokaryotic (bacterial cells), are standard procedures well known in the art.

Vectors

There is a wide array of vectors known and available in the art that are useful for the expression of differentially expressed nucleic acid molecules according to the invention. The selection of a particular vector clearly depends upon the intended use the polypeptide encoded by the differentially expressed nucleic acid. For example, the selected vector must be capable of driving expression of the polypeptide in the desired cell type, whether that cell type be prokaryotic or eukaryotic. Many vectors comprise sequences allowing both prokaryotic vector replication and eukaryotic expression of operably linked gene sequences.

Vectors useful according to the invention may be autonomously replicating, that is, the vector, for example, a plasmid, exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors.

Vectors useful according to the invention preferably comprise sequences operably linked to the sequence of interest (e.g., Reg1α) that permit the transcription and translation of the sequence. Sequences that permit the transcription of the linked sequence of interest include a promoter and optionally also include an enhancer element or elements permitting the strong expression of the linked sequences. The term “transcriptional regulatory sequences” refers to the combination of a promoter and any additional sequences conferring desired expression characteristics (e.g., high level expression, inducible expression, tissue- or cell-type-specific expression) on an operably linked nucleic acid sequence.

The selected promoter may be any DNA sequence that exhibits transcriptional activity in the selected host cell, and may be derived from a gene normally expressed in the host cell or from a gene normally expressed in other cells or organisms. Examples of promoters include, but are not limited to the following: A) prokaryotic promoters—E. coli lac, tac, or trp promoters, lambda phage P_{R} or P_{L} promoters, bacteriophage T7, T3, Sp6 promoters, B. subtilis alkaline protease promoter, and the B. stearothermophilus maltogenic amylase promoter, etc.; B) eukaryotic promoters—yeast promoters, such as GAL1, GAL4 and other glycolytic gene promoters (see for example, Hitzeman et al., 1980, J. Biol. Chem. 255: 12073-12080; Alber & Kawasaki, 1982, J. Mol. Appl. Gen. 1: 419-434), LEU2 promoter (Martinez-Garcia et al., 1989, Mol Gen Genet. 217: 464-470), alcohol dehydrogenase gene promoters (Young et al., 1982, in Genetic Engineering of Microorganisms for Chemicals, Hollaender et al., eds., Plenum Press, NY), or the TPI1 promoter (U.S. Pat. No. 4,599,311); insect promoters, such as the polyhedrin promoter (U.S. Pat. No. 4,745,051; Vasuvedan et al., 1992, FEBS Lett. 311: 7-11), the P10 promoter (Vlak et al., 1988, J. Gen. Virol. 69: 765-776), the Autographa californica polyhedrosis virus basic protein promoter (EP 397485), the baculovirus immediate-early gene promoter gene 1 promoter (U.S. Pat. Nos. 5,155,037 and 5,162,222), the baculovirus 39K delayed-early gene promoter (also U.S. Pat. Nos. 5,155,037 and 5,162,222) and the OpMNPV immediate early promoter 2; mammalian promoters—the SV40 promoter (Subramani et al., 1981, Mol. Cell. Biol. 1: 854-864), metallothionein promoter (MT-1; Palmiter et al., 1983, Science 222: 809-814), adenovirus 2 major late promoter (Yu et al., 1984, Nucl. Acids Res. 12: 9309-21), cytomegalovirus (CMV) or other viral promoter (Tong et al., 1998, Anticancer Res. 18: 719-725), or even the endogenous promoter of a gene of interest in a particular cell type.

A selected promoter may also be linked to sequences rendering it inducible or tissue-specific. For example, the addition of a tissue-specific enhancer element upstream of a selected promoter may render the promoter more active in a given tissue or cell type. Alternatively, or in addition, inducible expression may be achieved by linking the promoter to any of a number of sequence elements permitting induction by, for example, thermal changes (temperature sensitive), chemical treatment (for example, metal ion- or IPTG-inducible), or the addition of an antibiotic inducing agent (for example, tetracycline).

Regulatable expression is achieved using, for example, expression systems that are drug inducible (e.g., tetracycline, rapamycin or hormone-inducible). Drug-regulatable promoters that are particularly well suited for use in mammalian cells include the tetracycline regulatable promoters, and glucocorticoid steroid-, sex hormone steroid-, ecdysone-, lipopolysaccharide (LPS)- and isopropylthiogalactoside (IPTG)-regulatable promoters. A regulatable expression system for use in mammalian cells should ideally, but not necessarily, involve a transcriptional regulator that binds (or fails to bind) nonmammalian DNA motifs in response to a regulatory agent, and a regulatory sequence that is responsive only to this transcriptional regulator.

Tissue-specific promoters may also be used to advantage in differentially expressed sequence-encoding constructs of the invention. A wide variety of tissue-specific promoters is known. As used herein, the term “tissue-specific” means that a given promoter is transcriptionally active (i.e., directs the expression of linked sequences sufficient to permit detection of the polypeptide product of the promoter) in less than all cells or tissues of an organism. A tissue specific promoter is preferably active in only one cell type, but may, for example, be active in a particular class or lineage of cell types (e.g., hematopoietic cells). A tissue specific promoter useful according to the invention comprises those sequences necessary and sufficient for the expression of an operably linked nucleic acid sequence in a manner or pattern that is essentially the same as the manner or pattern of expression of the gene linked to that promoter in nature. The following is a non-exclusive list of tissue specific promoters and literature references containing the necessary sequences to achieve expression characteristic of those promoters in their respective tissues; the entire content of each of these literature references is incorporated herein by reference. Examples of tissue specific promoters useful in the present invention are as follows:

Bowman et al., 1995 Proc. Natl. Acad. Sci. USA 92,12115-12119 describe a brain-specific transferrin promoter; the synapsin I promoter is neuron specific (Schoch et al., 1996 J. Biol. Chem. 271, 3317-3323); the nestin promoter is post-mitotic neuron specific (Uetsuki et al., 1996 J. Biol. Chem. 271, 918-924); the neurofilament light promoter is neuron specific (Charron et al., 1995 J. Biol. Chem. 270, 30604-30610); the acetylcholine receptor promoter is neuron specific (Wood et al., 1995 J. Biol. Chem. 270, 30933-30940); and the potassium channel promoter is high-frequency firing neuron specific (Gan et al., 1996 J. Biol. Chem 271, 5859-5865). Any tissue specific transcriptional regulatory sequence known in the art may be used to advantage with a vector encoding a differentially expressed nucleic acid sequence obtained from an animal subjected to pain.

In addition to promoter/enhancer elements, vectors useful according to the invention may further comprise a suitable terminator. Such terminators include, for example, the human growth hormone terminator (Palmiter et al., 1983, supra), or, for yeast or fungal hosts, the TPI1 (Alber & Kawasaki, 1982, supra) or ADH3 terminator (McKnight et al., 1985, EMBO J. 4: 2093-2099).

Vectors useful according to the invention may also comprise polyadenylation sequences (e.g., the SV40 or Ad5E1b poly(A) sequence), and translational enhancer sequences (e.g., those from Adenovirus VA RNAs). Further, a vector useful according to the invention may encode a signal sequence directing the recombinant polypeptide to a particular cellular compartment or, alternatively, may encode a signal directing secretion of the recombinant polypeptide.

a. Plasmid Vectors.

Any plasmid vector that allows expression of a coding sequence of interest (e.g., the coding sequence of Reg1α) in a selected host cell type is acceptable for use according to the invention. A plasmid vector useful in the invention may have any or all of the above-noted characteristics of vectors useful according to the invention. Plasmid vectors useful according to the invention include, but are not limited to the following examples: Bacterial—pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, and pRIT5 (Pharmacia); Eukaryotic—pWLneo, pSV2cat, pOG44, pXT1, pSG (Stratagene) pSVK3, pBPV, pMSG, and pSVL (Pharmacia). However, any other plasmid or vector may be used as long as it is replicable and viable in the host.

b. Bacteriophage Vectors.

There are a number of well known bacteriophage-derived vectors useful according to the invention. Foremost among these are the lambda-based vectors, such as Lambda Zap II or Lambda-Zap Express vectors (Stratagene) that allow inducible expression of the polypeptide encoded by the insert. Others include filamentous bacteriophage such as the M13-based family of vectors.

c. Viral Vectors.

A number of different viral vectors are useful according to the invention, and any viral vector that permits the introduction and expression of one or more of the polynucleotides of the invention in cells is acceptable for use in the methods of the invention. Viral vectors that can be used to deliver foreign nucleic acid into cells include but are not limited to retroviral vectors, adenoviral vectors, adeno-associated viral vectors, herpesviral vectors, and Semliki forest viral (alphaviral) vectors. Defective retroviruses are well characterized for use in gene transfer (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14, and other standard laboratory manuals.

In addition to retroviral vectors, Adenovirus can be manipulated such that it encodes and expresses a gene product of interest but is inactivated in terms of its ability to replicate in a normal lytic viral life cycle (see for example Berkner et al., 1988, BioTechniques 6:616; Rosenfeld et al., 1991, Science 252:431-434; and Rosenfeld et al., 1992, Cell 68:143-155). Suitable adenoviral vectors derived from the adenovirus strain Ad type 5 d1324 or other strains of adenovirus (e.g., Ad2, Ad3, Ad7 etc.) are well known to those skilled in the art. Adeno-associated virus (AAV) is a naturally occurring defective virus that requires another virus, such as an adenovirus or a herpes virus, as a helper virus for efficient replication and a productive life cycle. (For a review see Muzyczka et al., 1992, Curr. Topics in Micro. and Immunol. 158:97-129). An AAV vector such as that described in Traschin et al. (1985, Mol. Cell. Biol. 5:3251-3260) can be used to introduce nucleic acid into cells. A variety of nucleic acids have been introduced into different cell types using AAV vectors (see, for example, Hermonat et al., 1984, Proc. Natl. Acad. Sci. USA 81: 6466-6470; and Traschin et al., 1985, Mol. Cell. Biol. 4: 2072-2081).

Host Cells

Any cell into which a recombinant vector carrying a gene of interest (e.g., a sequence encoding Reg1α) may be introduced and wherein the vector is permitted to drive the expression of the peptide encoded by the differentially expressed sequence is useful according to the invention. Any cell in which a differentially expressed molecule of the invention may be expressed and preferably detected is a suitable host, wherein the host cell is preferably a mammalian cell and more preferably a human cell. Vectors suitable for the introduction of nucleic acid sequences to host cells from a variety of different organisms, both prokaryotic and eukaryotic, are described herein above or known to those skilled in the art.

Host cells may be prokaryotic, such as any of a number of bacterial strains, or may be eukaryotic, such as yeast or other fungal cells, insect or amphibian cells, or mammalian cells including, for example, rodent, simian or human cells. Cells may be primary cultured cells, for example, primary human fibroblasts or keratinocytes, or may be an established cell line, such as NIH3T3, 293T or CHO cells. Further, mammalian cells useful in the present invention may be phenotypically normal or oncogenically transformed. It is assumed that one skilled in the art can readily establish and maintain a chosen host cell type in culture.

Introduction of Vectors to Host Cells.

Vectors useful in the present invention may be introduced to selected host cells by any of a number of suitable methods known to those skilled in the art. For example, vector constructs may be introduced to appropriate bacterial cells by infection, in the case of E. coli bacteriophage vector particles such as lambda or M13, or by any of a number of transformation methods for plasmid vectors or for bacteriophage DNA. For example, standard calcium-chloride-mediated bacterial transformation is still commonly used to introduce naked DNA to bacteria (Sambrook et al., 1989, Molecular Cloning, A Laboratory Manual, Cold Spring Harbor Laboratory Press, Cold Spring Harbor, N.Y.), but electroporation may also be used (Ausubel et al., 1988, Current Protocols in Molecular Biology, (John Wiley & Sons, Inc., NY, N.Y.)).

For the introduction of vector constructs to yeast or other fungal cells, chemical transformation methods are generally used (e.g. as described by Rose et al., 1990, Methods in Yeast Genetics, Cold Spring Harbor Laboratory Press, Cold Spring Harbor, N.Y.). For transformation of S. cerevisiae, for example, the cells are treated with lithium acetate to achieve transformation efficiencies of approximately 10⁴ colony-forming units (transformed cells)/μg of DNA. Transformed cells are then isolated on selective media appropriate to the selectable marker used. Alternatively, or in addition, plates or filters lifted from plates may be scanned for GFP fluorescence to identify transformed clones.

For the introduction of vectors comprising a sequence of interest to mammalian cells, the method used will depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation. These methods are detailed, for example, in Current Protocols in Molecular Biology (Ausubel et al., 1988, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

Following transfection with a vector of the invention, eukaryotic (e.g., human) cells successfully incorporating the construct (intra- or extrachromosomally) may be selected, as noted above, by either treatment of the transfected population with a selection agent, such as an antibiotic whose resistance gene is encoded by the vector, or by direct screening using, for example, FACS of the cell population or fluorescence scanning of adherent cultures. Frequently, both types of screening may be used, wherein a negative selection is used to enrich for cells taking up the construct and FACS or fluorescence scanning is used to further enrich for cells expressing differentially expressed polynucleotides or to identify specific clones of cells, respectively. For example, a negative selection with the neomycin analog G418 (Life Technologies, Inc.) may be used to identify cells that have received the vector, and fluorescence scanning may be used to identify those cells or clones of cells that express the vector construct to the greatest extent.

Reg1α and TIMP1 Polypeptides

The present invention provides a method for the detection of colorectal cancer in an individual by detecting the presence of Reg1α or TIMP1 in a clinical sample from an individual. In addition the invention encompasses the detection of cancer by identifying Reg1α or TIMP1 gene product in colon tissue or cells. Alternatively, the invention relates to a method for the detection of colorectal cancer in an individual wherein colorectal cancer is identified by detecting the presence of Reg1α or TIMP1 and at least one additional colorectal cancer associated marker in the clinical sample from an individual. Polypeptides of the present invention, the detection of which is indicative of colorectal cancer include those having the sequence shown in one or more of SEQ ID Nos. 2, 4, or 100, or alternatively, which are encoded by one or more of SEQ ID Nos. 1, 3 or 33.

Preferred polypeptides which can be detected and are thus indicative of colorectal cancer in an individual are those that are encoded by nucleic acid sequences at least about 70%, 75%, 80%, 90%, 95%, 97%, or 98% identical to a mRNA sequence complementary to the nucleic acid sequence of SEQ ID Nos. 1, 3 or 33. Particularly preferred polypeptides are those of SEQ ID Nos. 2, 4, or 99, or fragments thereof, or polypeptide sequences which are at least about 70%, 75%, 80%, 90%, 95%, 98% or 99% identical in sequence to the amino acid sequence of one or more of SEQ ID Nos. 2, 4, or 100.

In addition to a method for detecting colorectal cancer by identifying the presence of the Reg1α or TIMP1 polypeptide in a clinical sample from an individual, the invention further comprises a method of detecting cancer by identifying the presence of Reg1α or TIMP1 in addition to at least one other colorectal cancer associated marker in the same sample (e.g., in the same serum, tissue, or cell sample).

Antibodies

The invention provides a method for colorectal cancer detection comprising the step of detecting the presence of Reg1α or TIMP1 (and optionally, at least one additional colorectal cancer associated marker) in a clinical sample from an individual. In one embodiment, the presence of Reg1α or TIMP1, or other marker, in such a sample is detected using a polypeptide ligand which is preferably detectably labeled, and is capable of binding to Reg1α or TIMP1, and if present, the other marker, in the sample. In a preferred embodiment, the polypeptide ligand is an antibody. Antibodies of the invention include, but are not limited to, polyclonal, monoclonal, multispecific, human, humanized, or chimeric antibodies, single chain antibodies, Fab fragments, Fv fragments F(ab′) fragments, fragments produced by a Fab expression library, anti-iodiotypic antibodies, or other epitope binding polypeptide. Preferably, an antibody, useful in the present invention for the detection of Reg1α or TIMP1 (and optionally at least one additional colorectal cancer associated marker), is a human antibody or fragment thereof, including scFv, Fab, Fab′, F(ab′), Fd, single chain antibody, of Fv. Antibodies, useful in the invention may include a complete heavy or light chain constant region, or a portion thereof, or an absence thereof. An antibody, useful in the invention, may be obtained from an art recognized host, such as rabbit, mouse, rat, donkey, sheep, goat, guinea pig, camel, horse, or chicken. In one embodiment, an antibody, useful in the invention can be a humanized antibody, in which amino acids have been replaced in the non-antigen binding regions in order to more closely resemble a human antibody, while still retaining the original binding ability. Methods for making humanized antibodies are described in Teng et al., 1983, Proc. Natl. Acad. Sci. USA 80: 7308-7312; Kozbor et al., 1983, Immunology Today 4: 7279; Olsson et al., 1982, Meth. Enzymol. 92: 3-16; WO 92/06193; EP 0239400.

Antibodies of the present invention may be monospecific, dispecific, trispecific, or of greater multispecificity. As such, Reg1α or TIMP1 and optionally an additional colorectal cancer associated marker useful for the detection of colorectal cancer may be detected with separate antibodies, or may be detected with the same antibody. Alternatively, a multispecific antibody may exhibit different specificities for different epitopes on the same protein (e.g., different epitopes on Reg1α). While specificity of an antibody useful in the present invention to either Reg1α or one or more additional colorectal cancer associated markers is preferred, antibodies that bind polypeptides with at least 95%, 90%, 85%, 75%, 65%, 55%, and at least 50% identity to a polypeptide useful in the present invention for the detection of colorectal cancer (i.e., Reg1α, and/or an additional colorectal cancer associated marker) are also included in the present invention. Also encompassed in the present invention are antibodies which bind to polypeptide molecules which are encoded by one or more nucleic acid sequences which are complementary to, or hybridize to the sequences of SEQ ID Nos. 1, 3 or 33, or one or more sequences which are complementary to, or hybridize to a nucleic acid sequence which encodes an additional colorectal cancer associated marker as described herein.

Antibodies of the present invention which are useful for the detection of colorectal cancer may further act as agonists or antagonists of the activity of the polypeptide molecules to which they bind, and may thus be useful as therapeutic molecules for the treatment or prevention of colorectal cancer.

An important, but not limiting, role of an antibody of the present invention is to provide for the purification, or detection of Reg1α or TIMP1 or other colorectal cancer associated markers in a patient sample, including both in vitro and in vivo detection methods. Antibodies useful for the detection of colorectal cancer as described herein do not have to be used alone, and can be fused to other polypeptides, including a heterologous polypeptide at the N- or C-terminus of the antibody polypeptide sequence. For example, an antibody useful in the present invention may be fused with a detectable label to facilitate detection of the antibody when bound to a target polypeptide. Methods for detectably labeling an antibody polypeptide are known to those of skill in the art.

For the production of antibodies useful in the present invention, various hosts including goats, rabbits, rats, mice, etc., may be immunized by injection with the protein products (or any portion, fragment, or oligonucleotide thereof which retains immunogenic properties) of the candidate genes of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Polyclonal antisera or monoclonal antibodies can be made using methods known in the art. A mammal such as a mouse, hamster, or rabbit, can be immunized with an immunogenic form of a Reg1α or TIMP1 polypeptide, fragment, modified form thereof, or variant form thereof. Alternatively, an animal may be immunized with an immunogenic form of one or more additional colorectal cancer associated marker polypeptides. Techniques for conferring immunogenicity on such molecules include conjugation to carriers or other techniques well known in the art. For example, the immunogenic molecule can be administered in the presence of adjuvant as described above. Immunization can be monitored by detection of antibody titers in plasma or serum. Standard immunoassay procedures can be used with the immunogen as antigen to assess the levels and the specificity of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (see, e.g., Kohler and Milstein, 1975, Nature 256: 495-497; Kozbor et al., 1983, Immunol. Today 4: 72, Cole et al., 1985, In Monoclonal Antibodies in Cancer Therapy, Allen R. Bliss, Inc., pages 77-96). Additionally, techniques described for the production of single-chain antibodies (U.S. Pat. No. 4,946,778) can be adapted to produce antibodies according to the invention.

Antibody fragments which can specifically bind to a polypeptide of the invention such as Reg1α or TIMP1 or other colorectal cancer associated marker polypeptides, fragments thereof, modified forms thereof, and variants thereof, also may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. VH regions and FV regions can be expressed in bacteria using phage expression libraries (e.g., Ward et al., 1989, Nature 341: 544-546; Huse et al., 1989, Science 246: 1275-1281; McCafferty et al., 1990, Nature 348: 552-554).

Chimeric antibodies, i.e., antibody molecules that combine a non-human animal variable region and a human constant region also are within the scope of the invention. Chimeric antibody molecules include, for example, the antigen binding domain from an antibody of a mouse, rat, or other species, with human constant regions. Standard methods may be used to make chimeric antibodies containing the immunoglobulin variable region which recognizes the gene product of Reg1α antigens of the invention (see, e.g., Morrison et al., 1985, Proc. Natl. Acad. Sci. USA 81: 6851; Takeda et al., 1985, Nature 314: 452; U.S. Pat. No. 4,816,567; U.S. Pat. No. 4,816,397).

Other Colorectal Cancer Specific Analysis

In addition to the detection of colorectal cancer by identifying expression of Reg1α or TIMP1, or detecting Reg1α or TIMP1 polypeptides, the present invention further comprises a method for detecting colorectal cancer wherein a nucleic acid molecule encoding Reg1α or TIMP1, or Reg1α or TIMP1 polypeptide is identified in combination with at least one other nucleic acid sequence encoding a known colorectal cancer associated marker in a clinical sample from an individual. Alternatively, the presence of Reg1α or TIMP1 is detected in combination with at least one additional colorectal cancer marker amino acid sequence. Similar to the methods described above for Reg1α, a nucleic acid molecule which encodes at least one other colorectal cancer associated marker may be used to generate a nucleic acid probe for detection of the colorectal cancer associated marker sequence in a patient sample, or may be used to generate amplification primers to amplify the colorectal cancer associated marker sequence from a patient sample comprising the sequence, thus identifying the presence of the colorectal cancer associated marker in the sample, and thus indicating the detection of colorectal cancer. A colorectal cancer associated marker polypeptide sequence may be used, as described above for Reg1α to generate antibodies useful for detection of the colorectal cancer associated marker in a clinical sample. Methods for detecting a colorectal cancer associated marker nucleic acid or amino acid sequence are described below, and may be adapted from the methods for the detection of Reg1α nucleic acid or amino acid in a clinical sample.

A “colorectal cancer associated marker” useful in the present invention, refers to a polypeptide or nucleic acid sequence which exhibits over- or underexpression of at least 10% in colorectal cancer cells, tissue, or serum obtained from an individual having colorectal cancer, relative to the level of expression in cells, tissue, or serum obtained from an individual that does not have colorectal cancer. Non-limiting examples of colorectal cancer associated markers useful in the present invention include the nucleic acid molecules of SEQ ID Nos 1, 3, 5-71, and/or the polypeptide molecules of SEQ ID Nos 2, 4, 72-138. In one embodiment, the polypeptide sequences of SEQ ID Nos 2, 4, 72-138 are encoded by the nucleic acid sequences of 1, 3, 5-71, respectively. It will be appreciated by one of skill in the art that, where the method of the invention relates to detection of Reg1α and at least one other colorectal cancer associated marker, TIMP1 may be included as a potential “other colorectal cancer associated marker”. Likewise, where the detection method is based on the detection of TIMP1 and at least one other colorectal cancer associated marker, Reg1α may be included as a potential “other colorectal cancer associated marker”. Alternatively, a colorectal cancer associated marker, as used in the present invention, may refer to a carbohydrate epitope present on a polypeptide or nucleic acid molecule and/or an antibody molecule which recognizes and is capable of binding to such an epitope, wherein the carbohydrate epitope is known to be associated with the presence of colorectal cancer in an individual. Such carbohydrate epitopes may be present on more than one unrelated protein or polypeptide. In one embodiment, such a carbohydrate epitope is CA 19-9, also known as sialyl-Lewis^{a}, is a tumor marker defined by a monoclonal antibody as a carbohydrate epitope, related to the blood group antigens, composed of a branching, 5-sugar structure covalently bound to a variety of glycoproteins or glycolipids. The proteins primarily belong to the mucin family and the lipids are usually membrane associated. The CA 19-9 epitope is typically the terminal moiety of a complex, O-linked carbohydrate structure on either macromolecule. Other tumor markers also defined as various carbohydrate epitopes useful in the present invention as a “colorectal cancer associated marker” include CA72-4 which is indicative of the presence of the Tag 72 antigen, which is a triply sialylated Tn antigen on varing protein backbones; Thomsen Freidenreich antigen (TF), which is a sialylated n-acetyl galactosamine moeity O-linked to various peptides; Tn and sialyated Tn (sTn) which is the backbone of the TF antigen without the terminal n-acetyl galactosamine moeity, O-linked to various peptides; CA 50 which is an epitope corresponding to sialylated Lewis A blood group antigen; CA 549 which is a CHO moiety on muc-1; CA 242 which is a sialylated CHO; LASA which is a lipid associated sialic acid, that is, a lipid without a protein associated to it; Du-PAN's 1-5, which are pancreatic associated mucin-like CHO antigens. These useful colon cancer specific antigens and others are known in the art and are described, for example, in “Serological Cancer Markers” Sell, S., Ed. 1992. Humana Press Inc., Totowa, N.J.

Table 1 below shows a list of “colorectal cancer associated markers” useful in the invention (although colorectal cancer associated markers useful in the invention are not limited to those shown in Table 1), and there correspondence with the sequences set forth in the “Sequence listing”.

TABLE 1

| SEQ ID | Gene | | |
|---|---|---|---|
| NO | Symbol | Length | Type |
| 5 | CEACAM5 | 2974 | DNA |
| 6 | AFP | 2032 | DNA |
| 7 | IL8 | 1639 | DNA |
| 8 | SPP1 | 1524 | DNA |
| 9 | KIAA1077 | 5500 | DNA |
| 10 | MMP12 | 1778 | DNA |
| 11 | UBD | 777 | DNA |
| 12 | COL1A1 | 5921 | DNA |
| 13 | LUM | 1804 | DNA |
| 14 | ENC1 | 4827 | DNA |
| 15 | PIGPC1 | 1098 | DNA |
| 16 | GTF3A | 1381 | DNA |
| 17 | CTSB | 1978 | DNA |
| 18 | MCJ | 1074 | DNA |
| 19 | SLC12A2 | 4098 | DNA |
| 20 | C20orf42 | 3120 | DNA |
| 21 | SDBCAG84 | 1337 | DNA |
| 22 | NAP1L1 | 2908 | DNA |
| 23 | OSF-2 | 3213 | DNA |
| 24 | COL6A3 | 10558 | DNA |
| 25 | SPARC | 2133 | DNA |
| 26 | TGFBI | 2691 | DNA |
| 27 | FN1 | 8027 | DNA |
| 28 | COL1A2 | 5084 | DNA |
| 29 | S100A11 | 595 | DNA |
| 30 | LC27 | 2116 | DNA |
| 31 | IRAK1 | 3583 | DNA |
| 32 | IFITM2 | 905 | DNA |
| 33 | TIMP1 | 782 | DNA |
| 34 | IGFBP7 | 1124 | DNA |
| 35 | IFITM1 | 647 | DNA |
| 36 | COL3A1 | 5489 | DNA |
| 37 | IGFBP5 | 1722 | DNA |
| 38 | RegIV | 1200 | DNA |
| 39 | AGR2 | 1701 | DNA |
| 40 | HSPCA | 2259 | DNA |
| 41 | KIAA1199 | 7080 | DNA |
| 42 | MMP1 | 1973 | DNA |
| 43 | MMP7 | 1127 | DNA |
| 44 | TSC | 1163 | DNA |
| 45 | HAIK1 | 2007 | DNA |
| 46 | DAP3 | 1650 | DNA |
| 47 | | 2566 | DNA |
| 48 | | 2067 | DNA |
| 49 | KRT8 | 1752 | DNA |
| 50 | KRT18 | 1412 | DNA |
| 51 | KRT19 | 1407 | DNA |
| 52 | KRT20 | 1723 | DNA |
| 53 | MUC1 | 4139 | DNA |
| 54 | MUC2 | 15720 | DNA |
| 55 | MUC3 | 4707 | DNA |
| 56 | MUC5AC | 4151 | DNA |
| 57 | CGB5 | 880 | DNA |
| 58 | EGFR | 5532 | DNA |
| 59 | ERBB2 | 4530 | DNA |
| 60 | FTH1 | 801 | DNA |
| 61 | FTL | 878 | DNA |
| 62 | ALPP | 2747 | DNA |
| 63 | ODC1 | 2062 | DNA |
| 64 | MUC16 | 3557 | DNA |
| 65 | CEACAM1 | 3464 | DNA |
| 66 | CEACAM3 | 1022 | DNA |
| 67 | CEACAM4 | 1190 | DNA |
| 68 | CEACAM6 | 2249 | DNA |
| 69 | CEACAM7 | 2292 | DNA |
| 70 | CEACAM8 | 2297 | DNA |
| 71 | CA9 | 1552 | DNA |
| 72 | CEACAM5 | 702 | Protein |
| 73 | AFP | 609 | Protein |
| 74 | IL8 | 99 | Protein |
| 75 | SPP1 | 300 | Protein |
| 76 | KIAA1077 | 871 | Protein |
| 77 | MMP12 | 470 | Protein |
| 78 | UBD | 165 | Protein |
| 79 | COL1A1 | 1464 | Protein |
| 80 | LUM | 338 | Protein |
| 81 | ENC1 | 589 | Protein |
| 82 | PIGPC1 | 193 | Protein |
| 83 | GTF3A | 423 | Protein |
| 84 | CTSB | 339 | Protein |
| 85 | MCJ | 150 | Protein |
| 86 | SLC12A2 | 1212 | Protein |
| 87 | C20orf42 | 230 | Protein |
| 88 | SDBCAG84 | 383 | Protein |
| 89 | NAP1L1 | 391 | Protein |
| 90 | OSF-2 | 836 | Protein |
| 91 | COL6A3 | 3176 | Protein |
| 92 | SPARC | 303 | Protein |
| 93 | TGFBI | 683 | Protein |
| 94 | FN1 | 2355 | Protein |
| 95 | COL1A2 | 1366 | Protein |
| 96 | S100A11 | 105 | Protein |
| 97 | LC27 | 283 | Protein |
| 98 | IRAK1 | 712 | Protein |
| 99 | IFITM2 | 132 | Protein |
| 100 | TIMP1 | 207 | Protein |
| 101 | IGFBP7 | 282 | Protein |
| 102 | IFITM1 | 125 | Protein |
| 103 | COL3A1 | 1466 | Protein |
| 104 | IGFBP5 | 272 | Protein |
| 105 | RegIV | 158 | Protein |
| 106 | AGR2 | 175 | Protein |
| 107 | HSPCA | 732 | Protein |
| 108 | KIAA1199 | 1361 | Protein |
| 109 | MMP1 | 469 | Protein |
| 110 | MMP7 | 267 | Protein |
| 111 | TSC | 216 | Protein |
| 112 | HAIK1 | 422 | Protein |
| 113 | DAP3 | 398 | Protein |
| 114 | | 75 | Protein |
| 115 | | 163 | Protein |
| 116 | KRT8 | 483 | Protein |
| 117 | KRT18 | 430 | Protein |
| 118 | KRT19 | 400 | Protein |
| 119 | KRT20 | 424 | Protein |
| 120 | MUC1 | 1255 | Protein |
| 121 | MUC2 | 5179 | Protein |
| 122 | MUC3 | 1217 | Protein |
| 123 | MUC5AC | 1373 | Protein |
| 124 | CGB5 | 165 | Protein |
| 125 | EGFR | 1210 | Protein |
| 126 | ERBB2 | 1255 | Protein |
| 127 | FTH1 | 190 | Protein |
| 128 | FTL | 175 | Protein |
| 129 | ALPP | 535 | Protein |
| 130 | ODC1 | 461 | Protein |
| 131 | MUC16 | 1148 | Protein |
| 132 | CEACAM1 | 526 | Protein |
| 133 | CEACAM3 | 212 | Protein |
| 134 | CEACAM4 | 244 | Protein |
| 135 | CEACAM6 | 344 | Protein |
| 136 | CEACAM7 | 265 | Protein |
| 137 | CEACAM8 | 349 | Protein |
| 138 | CA9 | 459 | Protein |

Detection Assays

The present invention provides method for detecting colorectal cancer, or alternatively, determining whether a subject is at risk for developing colorectal cancer by detecting the disclosed biomarkers (i.e., the nucleic acid sequence of Reg1α or TIMP1 and optionally, one or more nucleic acid sequences encoding an additional colorectal cancer associated marker and/or polypeptide markers such as Reg1α or TIMP1 and optionally, at least one additional colorectal cancer associated marker) for the disease or condition encoded thereby.

In clinical applications, human tissue samples, preferably serum, can be screened for the presence and/or absence of Reg1α or TIMP1 and/or other colorectal cancer associated markers identified herein. Such samples may comprise tissue samples, whole cells, cell lysates, or isolated nucleic acids, including, for example, needle biopsy cores, surgical resection samples, lymph node tissue, or serum. A sample for analysis as described herein is preferably a serum sample. A serum sample may be obtained from an individual using methods which are well known to those of skill in the art. Briefly, a whole venous or arterial blood sample from an individual is collected into a test tube. The whole blood sample is permitted to incubate at room temperature for approximately 15-30 to allow the blood to clot. Once clotted, the sample is centrifuged at approximately 1500 to 3000 rpm for 5-30 minutes to completely separate the serum from the cellular components. This centrifugation may be repeated if necessary to achieve complete separation. The resulting serum sample may be subsequently screened for the presence of Reg1α nucleic acid or amino acid and/or one or more additional colorectal cancer associated markers as described herein.

Screening for Nucleic Acid Molecules

In one embodiment, the detection method of the present invention comprises determining whether a clinical sample from an individual contains mRNA of a colorectal cancer associated marker, preferably Reg1α or TIMP1, but also optionally including additional colorectal cancer associated markers as described herein. Techniques for determining the presence of a nucleic acid molecule of interest include Northern blot analysis, reverse transcription-polymerase chain reaction (RT-PCR), in situ hybridization, PCR, and quantitative amplification.

Prior to detection of target nucleic acid molecules in a clinical sample, it is preferred to first isolate the mRNA from the sample to facilitate detection of the target sequence (i.e., a sequence encoding Reg1α or TIMP1). Methods for isolation of mRNA from a biological sample are well known in the art. Briefly, where the sample is a serum sample, for example, 0.1 ml of 2 M sodium acetate, pH 4, 1 ml water-saturated phenol, and 0.2 ml of 49:1 chloroform/isoamyl alcohol are added to the serum sample sequentially. The sample is mixed after the addition of each component, and incubated for 15 min at 0-4° C. after all components have been added. The sample is separated by centrifugation for 20 min at 10,000×g, 4° C., precipitated by the addition of 1 ml of 100% isopropanol, incubated for 30 minutes at −20° C. and pelleted by centrifugation for 10 minutes at 10,000×g, 4° C. The resulting RNA pellet is dissolved in 0.3 ml denaturing solution, transferred to a microfuge tube, precipitated by the addition of 0.3 ml of 100% isopropanol for 30 minutes at −20° C., and centrifuged for 10 minutes at 10,000×g at 4° C. The RNA pellet is washed in 70% ethanol, dried, and resuspended in 100-200 μl DEPC-treated water or DEPC-treated 0.5% SDS (Chomczynski and Sacchi, 1987, Anal. Biochem., 162: 156).

Alternatively, total RNA may be extracted from a clinical sample according to the present invention using a commercially available RNA isolation reagent such as Trizol (Invitrogen, Carlsbad, Calif.), following the manufacturers instructions. Purity and integrity of RNA is assessed by absorbance at 260/280 nm and separation of RNA samples on a 1% agarose gel followed by inspection under ultraviolet light.

Following mRNA isolation, the mRNA may be reverse transcribed to provide a cDNA sample according to methods well known to those of skill in the art (see, e.g., Ausubel et al. (1995), Short Protocols in Molecular Biology, 3^{rd} Ed. John Wiley and Sons, Inc.)

Accordingly, in one aspect, the invention provides probes and primers that specifically hybridize to the Reg1α or TIMP1 nucleic acid sequences disclosed herein, or which can hybridize to a nucleic acid molecule encoding an additional colorectal cancer associated marker as described herein. Accordingly, the nucleic acid probes comprise a region of a nucleic acid sequence of SEQ ID Nos 1, 3, or 33 sufficient to hybridize with a nucleic acid substantially complementary to the sequence of SEQ ID Nos 1, 3 or 33. Preferred nucleic acid molecules for use as probes/primers can further comprise a region of nucleic acid sequence substantially complementary to the sequence of SEQ ID Nos. 1, 3 or 33 sufficient to hybridize with the sequence of SEQ ID Nos. 1, 3 or 33. In addition, nucleic acid sequences useful as probes/primers comprise a nucleotide sequence at least about 8 nucleotides in length, at least about 12 nucleotides in length, preferably at least about 15 nucleotides, more preferably about 25 nucleotides, and most preferably at least 40 nucleotides, and up to all or nearly all of the coding sequence which is complementary to a portion of the coding sequence of a marker nucleic acid sequence, which nucleic acid sequence is represented by SEQ ID Nos: 1, 3 or 33, or a sequence complementary thereto.

In one embodiment, the method comprises using a nucleic acid probe to determine the presence of a Reg1α or TIMP1 nucleic acid molecule in a clinical sample (such as a serum sample or a nucleic acid sample extracted therefrom). Specifically, the method comprises:

1. Providing a nucleic acid probe comprising a nucleotide sequence at least about 8 nucleotides in length, at least about 12 nucleotides in length, preferably at least about 15 nucleotides, more preferably about 25 nucleotides, and most preferably at least about 40 nucleotides, and up to all or nearly all of the coding sequence which is complementary to a portion of the coding sequence of a nucleic acid sequence represented by SEQ ID Nos: 1, 3 or 33, or a sequence complementary thereto;

2. Obtaining a clinical sample from a patient potentially comprising a Reg1α or TIMP1 nucleic acid sequence;

3. Providing a second clinical sample from an individual known to not have colorectal cancer;

4. Contacting the nucleic acid probe under stringent conditions with RNA of each of said first and second clinical samples (e.g., in a Northern blot or in situ hybridization assay); and

5. Comparing (a) the amount of hybridization of the probe with RNA of the first serum sample, with (b) the amount of hybridization of the probe with RNA of the second clinical sample; wherein a statistically significant difference in the amount of hybridization with the RNA of the first clinical sample as compared to the amount of hybridization with the RNA of the second clinical sample is indicative of the presence of Reg1α or TIMP1 in the first clinical sample.

Although, primarily drawn to detection of Reg1α or TIMP1 in a clinical sample such as serum, in one aspect, the present invention provides a method comprising in situ hybridization detection of Reg1α or TIMP1 with a probe derived from a nucleic acid sequence represented by SEQ ID Nos: 1, 3 or 33, or a sequence complementary thereto. Preferably, the hybridization probe is detectably labeled. The method comprises contacting the labeled hybridization probe with a tissue or cell sample from an individual suspected of having colorectal cancer, washing off any unbound probe, and detecting the signal produced by the detectable label, wherein the detection of the detectable signal is indicative of the presence of Reg1α or TIMP1 in the sample, and thus permits the detection of colorectal cancer. Alternatively, the tissue or cell is additionally hybridized with a detectably labeled nucleic acid probe which is capable of specifically hybridizing with a nucleic acid sequence that encodes at least one additional colorectal cancer associated marker. Detection of the second detectably labeled probe is thus indicative of the presence of the additional colorectal cancer associated marker in the sample, and in conjunction with the detection of Reg1α or TIMP1, permits the detection of colorectal cancer in the individual. Specific methods for in situ hybridization are well known in the art.

Alternatively, methods such as PCR, Northern analysis, and Taqman may be used to detect and/or quantitate the expression of a nucleic acid sequence encoding Reg1α in a clinical sample. In one embodiment, reverse transcription PCR (RT-PCR) is performed using primers designed to specifically hybridize to a predetermined portion of the Reg1α mRNA sequence isolated from a clinical sample. Generation of a PCR product by such a reaction is thus indicative of the presence of the Reg1α or TIMP1 sequence in the sample. The technique of designing primers for PCR amplification is well known in the art. Oligonucleotide primers and probes are 5 to 100 nucleotides in length, ideally from 17 to 40 nucleotides, although primers and probes of different length are of use. Primers for amplification are preferably about 17-25 nucleotides. Primers useful according to the invention are also designed to have a particular melting temperature (Tm) by the method of melting temperature estimation. Commercial programs, including Oligo™ (MBI, Cascade, CO), Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65° C. and more preferably between about 50 and 60° C. Preferably, the Tm of a probe useful according to the invention is 7° C. higher than the Tm of the corresponding amplification primers. It is preferred that, following generation of cDNA by RT-PCR, the cDNA fragment is cloned into an appropriate sequencing vector, such as a PCRII vector (TA cloning kit; Invitrogen). The identity of each cloned fragment is then confirmed by sequencing in both directions. It is expected that the sequence obtained from sequencing would be the same as the known sequence of Reg1α to TIMP1 as described herein.

Alternatively, the presence of an mRNA sequence encoding Reg1α or TIMP1 may be detected by Northern analysis. Sequence confirmed cDNAs, that is, cDNAs encoding Reg1α or TIMP1 (or alternatively an additional colorectal cancer associated marker) are used to produce ³²P-labeled cDNA probes using techniques well known in the art (see, for example, Ausubel, supra). Labeled probes for Northern analysis may also be produced using commercially available kits (Prime-It Kit, Stratagene, La Jolla, Calif.). Northern analysis of total RNA obtained from a clinical sample may be performed using classically described techniques. For example, total RNA samples are denatured with formaldehyde/formamide and run for two hours in a 1% agarose, MOPS-acetate-EDTA gel. RNA is then transferred to nitrocellulose membrane by upward capillary action and fixed by UV cross-linkage. Membranes are pre-hybridized for at least 90 minutes and hybridized overnight at 42° C. Post hybridization washes are performed as known in the art (Ausubel, supra). The membrane is then exposed to x-ray film overnight with an intensifying screen at −80° C. Labeled membranes are then visualized after exposure to film. The signal produced on the x-ray film by the radiolabeled cDNA probes can then be quantified using any technique known in the art, such as scanning the film and quantifying the relative pixel intensity using a computer program such as NIH Image (National Institutes of Health, Bethesda, Md.), wherein the detection of hybridization of a Reg1α-specific probe to the clinical sample is indicative of the presence of Reg1α or TIMP1 and thus may be used to detect colorectal cancer.

In an alternate embodiment, the presence and optionally the quantity of Reg1α or TIMP1 in a clinical sample may be determined using the Taqman™ (Perkin-Elmer, Foster City, Calif.) technique, which is performed with a transcript-specific antisense probe (i.e., a probe capable of specifically hybridizing to Reg1α). This probe is specific for a Reg1α or TIMP1 PCR product and is prepared with a quencher and fluorescent reporter probe complexed to the 5′ end of the oligonucleotide. Different fluorescent markers can be attached to different reporters, allowing for measurement of two products in one reaction (e.g., measurement of Reg1α or TIMP1 and at least one additional colorectal cancer associated marker). When Taq DNA polymerase is activated, it cleaves off the fluorescent reporters by its 5′-to-3′ nucleolytic activity. The reporters, now free of the quenchers, fluoresce. The color change is proportional to the amount of each specific product and is measured by fluorometer; therefore, the amount of each color can be measured and the RT-PCR product can be quantified. The PCR reactions can be performed in 96 well plates so that samples derived from many individuals can be processed and measured simultaneously. The Taqman™ system has the additional advantage of not requiring gel electrophoresis and allows for quantification when used with a standard curve.

Screening for Polypeptide Molecules

The Reg1α- or TIMP1-specific and colorectal cancer marker-specific antibodies described above may be used to detect the presence of Reg1α or TIMP1 or an additional colorectal cancer associated marker in a clinical sample by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100,1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e. g., 1-4 hours) at 4 C, adding protein A and/or protein G sepharose beads to the cell lysate, incubating for about an hour or more at 4 C, washing the beads in lysis buffer and resuspending the beads in SDS/sample buffer. In the case of immunonprecipitation of a serum sample, however the above protocol is carried out absent the cell lysis step. The ability of the antibody to immunoprecipitate Reg1α or TIMP1 (or other colorectal cancer marker) antigen can be assessed by, e.g., western blot analysis. The parameters that can be modified to increase the binding of the antibody to an antigen and decrease the background (e.g., preclearing the cell lysate with sepharose beads) are well known to those of skill in the art (Ausubel et al, supra).

Reg1α or TIMP1 polypeptides, and optionally one or more additional colorectal cancer associated markers may be detected in a patient clinical sample using Western blot analysis. Briefly, Western blot analysis comprises preparing protein samples, electrophoresis of the protein samples in a polyacrylamide gel (e.g., 8%-20% SDS-PAGE), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an antihuman antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. Methods for the optimization of such an analysis are well known in the art (Ausubel, et al., supra).

Alternatively, the presence of Reg1α or TIMP1 and optionally one or more additional colorectal cancer associated markers in a clinical sample may be detected by ELISA. ELISAs comprise preparing antigen, coating the well of a 96 well microtiter plate (or other suitable container) with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest, that is, the antibody which will bind to Reg1α or TIMP1 or a second colorectal cancer associated marker) conjugated to a detectable compound may be added to the well. Further, instead of coating the well with the antigen, the antibody may be coated to the well. In this case, a second antibody conjugated to a detectable compound may be added following the addition of the antigen of interest to the coated well. This method may be modified or optimized according techniques which are known to those of skill in the art.

The binding affinity of an antibody to an antigen and the off-rate of an antibody antigen interaction can be determined by competitive binding assays. One example of such an assay is a radioimmunoassay comprising the incubation of labeled antigen (e.g., Reg1α labeled with 3H or 125I) with an anti-Reg1α or TIMP1 antibody in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Preferably, the above detection assays re be carried out using antibodies to detect the protein product encoded by a nucleic acid having the sequence of SEQ ID Nos: 1, 3 or 33, or a sequence complementary thereto. Preferably, the protein product has the sequence of one or more of SEQ ID Nos. 2, 4, or 100. In addition, the above detection assays may be conducted using one or more antibodies which specifically recognize and bind to at least one additional colorectal cancer associated marker. Accordingly, in one embodiment, the assay would include contacting the proteins of the test cell with an antibody specific for the gene product of a nucleic acid represented by SEQ ID Nos: 1, 3 or 33, or a sequence complementary thereto, and determining the approximate amount of immunocomplex formation by the antibody and the proteins of the test cell, wherein a detection of such an immunocomplex is indicative of the presence of the antigen, and thus, permits the detection of colorectal cancer.

Immunoassays, useful in the present invention include those described above, and can also include both homogeneous and heterogeneous procedures such as fluorescence polarization immunoassay (FPIA), fluorescence immunoassay (FIA), enzyme immunoassay (EIA), and nephelometric inhibition immunoassay (NIA).

In another embodiment, the level of the encoded product, i.e., the product encoded by SEQ ID Nos 1, 3 or 33, or a sequence complementary thereto, in a biological fluid (e.g., blood or urine) of a patient may be determined as a way of monitoring the level of expression of the marker nucleic acid sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of a hyperproliferative disorder, e.g., colorectal cancer. The level of the marker polypeptide can be used predictively to evaluate whether a sample of cells contains cells which are, or are predisposed towards becoming, transformed cells. Moreover, the subject method can be used to assess the phenotype of cells which are known to be transformed, the phenotyping results being useful in planning a particular therapeutic regimen. For instance, very high levels of the marker polypeptide in sample cells is a powerful diagnostic and prognostic marker for a cancer, such as colorectal cancer. The observation of marker polypeptide level can be utilized in decisions regarding, e.g., the use of more aggressive therapies.

As set out above, one aspect of the present invention relates to detection assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

Of particular importance to the subject invention is the ability to quantitate the level of normal or abnormal Reg1α or TIMP1 expression. The expression of Reg1α or TIMP1, and/or the level of expression of Reg1α or TIMP1 can be used predictively to evaluate whether a patient is predisposed towards developing colorectal cancer, or for determining the severity of colorectal cancer.

In one embodiment, tissue samples may be used to measure Reg1α or TIMP1 expression by immunohistochemical staining which may be used to determine the number of cells (i.e., colon cells) expressing Reg1α or TIMP1. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissue samples are fixed by treatment with a reagent such as formalin, glutaraldehyde, methanol, or the like. The samples are then incubated with an antibody, preferably a monoclonal antibody, with binding specificity for Reg1α or TIMP1 and optionally an additional colorectal cancer associated marker. This antibody may be conjugated to a label for subsequent detection of binding. Samples are incubated for a time sufficient for formation of the immunocomplexes. Binding of the antibody is then detected by virtue of a label conjugated to this antibody. Where the antibody is unlabeled, a second labeled antibody may be employed, e.g., which is specific for the isotype of the anti-marker polypeptide antibody. Examples of labels which may be employed include radionuclides, fluorescers, chemiluniinescers, enzymes and the like.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Other assays, known to those of skill in the art for determining the presence and/or quantity of a polypeptide in a sample (either serum or tissue) are also encompassed by the present invention.

Drug Screening

Several in vivo methods can be used to identify compounds that modulate expression of Reg1α or TIMP1 nucleic acids (SEQ ID Nos: 1, 3 or 33, or a sequence complementary thereto) and/or alter for example, inhibit the bioactivity of the encoded polypeptide (e.g., SEQ ID Nos: 2, 4, or 100).

Drug screening is performed by adding a test compound to a sample of cells, and monitoring the effect. A parallel sample which does not receive the test compound is also monitored as a control. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, and the ability of the cells to interact with other cells or compounds. Differences between treated and untreated cells indicates effects attributable to the test compound.

Desirable effects of a test compound include an effect on any phenotype that was conferred by the cancer-associated marker nucleic acid sequence. Examples include a test compound that limits the overabundance of mRNA, limits production of the encoded protein, or limits the functional effect of the protein. The effect of the test compound would be apparent when comparing results between treated and untreated cells.

The invention thus also encompasses methods of screening for agents which inhibit expression of Reg1α or TIMP1 nucleic acid (SEQ ID Nos: 1, 3 or 33, or a sequence complementary thereto) in vitro, comprising exposing either a cell or tissue in which Reg1α or TIMP1 nucleic acid mRNA is detectable or cultured cells comprising and capable of expressing Reg1α or TIMP1 nucleic acid to an agent in order to determine whether the agent is capable of inhibiting production of the mRNA; and determining the level of mRNA in the exposed cells or tissue, wherein a decrease in the level of the mRNA after exposure of the cell line to the agent is indicative of inhibition of the marker nucleic acid mRNA production.

Alternatively, the screening method may include in vitro screening of a cell or tissue in which Reg1α or TIMP1 is detectable, or cultured cells which express Reg1α or TIMP1, to an agent suspected of inhibiting production of Reg1α or TIMP1 protein; and determining the level of the Reg1α or TIMP1 protein in the cells or tissue, wherein a decrease in the level of marker protein after exposure of the cells or tissue to the agent is indicative of inhibition of marker protein production.

The invention also encompasses in vivo methods of screening for agents which inhibit expression of the marker nucleic acids, comprising exposing a mammal having tumor cells or serum in which Reg1α or TIMP1 mRNA or protein is detectable to an agent suspected of inhibiting production of marker mRNA or protein; and determining the level of marker mRNA or protein in serum or tumor cells of the exposed mammal. A decrease in the level of marker mRNA or protein after exposure of the mammal to the agent is indicative of inhibition of marker nucleic acid expression. Optionally, the effect of the candidate agent on the expression of at least one additional colorectal cancer associated marker may also be determined.

Accordingly, the invention provides a method comprising incubating a cell expressing the marker nucleic acids (SEQ ID Nos: 1, 3 or 33, or a sequence complementary thereto) with a test compound and measuring the mRNA or protein level. The invention further provides a method for quantitatively determining the level of expression of the marker nucleic acids in a cell population or clinical sample, and a method for determining whether an agent is capable of increasing or decreasing the level of expression of the Reg1α or TIMP1 nucleic acid in a cell population or clinical sample. The method for determining whether an agent is capable of increasing or decreasing the level of expression of Reg1α or TIMP1 nucleic acid in a cell population comprises the steps of (a) preparing cell extracts from control and agent-treated cell populations, (b) isolating the Reg1α or TIMP1 polypeptide from the cell extracts, (c) quantifying (e.g., in parallel) the amount of an immunocomplex formed between Reg1α or TIMP1 polypeptide and an antibody specific to said polypeptide. The Reg1α or TIMP1 polypeptide of this invention may also be quantified by assaying for its bioactivity. Agents that induce an increase in Reg1α or TIMP1 nucleic acid expression may be identified by their ability to increase the amount of immunocomplex formed in the treated cell as compared with the amount of the immunocomplex formed in the control cell. In a similar manner, agents that decrease expression of Reg1α or TIMP1 nucleic acid may be identified by their ability to decrease the amount of the immunocomplex formed in the treated cell extract as compared to the control cell.

mRNA levels can be determined by Northern blot hybridization. mRNA levels can also be determined by methods involving PCR. Other sensitive methods for measuring mRNA, which can be used in high throughput assays, e.g., a method using a DELFIA endpoint detection and quantification method, are described, e.g., in Webb and Hurskainen (1996) Journal of Biomolecular Screening 1:119. Reg1α protein levels can be determined by immunoprecipitations or immunohistochemistiy using an antibody that specifically recognizes the protein product of SEQ ID Nos: 2, 4, or 100.

Agents that are identified as active in the drug screening assay are candidates to be tested for their capacity to block cell proliferation activity. These agents would be useful for treating a disorder involving aberrant growth of cells, especially colon cells, especially colorectal cancer.

A variety of assay formats will suffice and, in light of the present disclosure, those not expressly described herein will nevertheless be comprehended by one of ordinary skill in the art. For instance, the assay can be generated in many different formats, and include assays based on cell-free systems, e.g., purified proteins or cell lysates, as well as cell-based assays which utilize intact cells.

In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, or with proteins purified or semi-purified from serum, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

### EXAMPLES

The examples below are non-limiting and are merely representative of various aspects and features of the present invention.

### Example 1

### Generation of anti-Reg1α Antibodies

To generate antibodies to Reg1α, the full-length open reading frame of Reg1α (shown in either SEQ ID NO: 1 or 3) was directionally cloned into a mammalian expression vector, such as pcDNA3.1N5-His (Invitrogen), which includes C-terminal epitope and purification tags. The insert sequence was verified by dideoxy sequencing (see, for example, Ausubel et al., Current Protocols in Molecular Biology, John Wiley and Sons). Recombinant fusion protein was produced in a transient expression system in mammalian cells (e.g. CHO cells). The recombinant protein was purified from the cell culture supernatants by immobilized metal affinity chromatography (IMAC) by utilizing the C terminal His-tag. The sequence of the Reg1α protein used for the production of antibodies of the present invention is shown in either of SEQ ID Nos 2 or 4, all of which represent a functional Reg1α protein, and which are encoded by SEQ ID Nos 1 or 3, respectively. The purified, recombinant Reg1α protein was emulsified in Freund's adjuvant and injected into rabbits. The animals were periodically boosted until they elicited a reasonable serum titer of specific antibody to Reg1a. Methods for antibody production are well known to those of skill in the art and may be found, for example, in Harlow et al. Antibodies: A laboratory manual, 1988, Cold Spring Harbor Laboratory. The polyclonal antibodies, which recognized both native and denatured Reg1α, were utilized to develop a microtiter-based ELISA assay. Methods of performing an ELISA assay are well known to those of skill in the art (see, for example, Asusbel et al., supra).

### Example 2

### Detection of Reg1α in Colorectal Cancer Patient Serum Samples

The present invention relates to a method for the detection of colorectal cancer in an individual, which method includes the detection of Reg1α polypeptides in a serum sample from an individual with colorectal cancer, wherein the detection of Reg1α is indicative of the presence of colorectal cancer. Accordingly, Reg1α expression was measured in serum samples obtained from patients having been diagnosed with colorectal cancer.

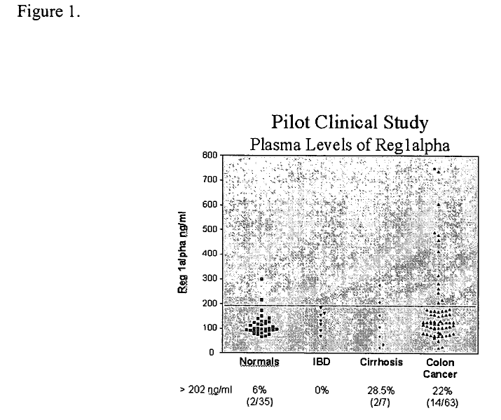

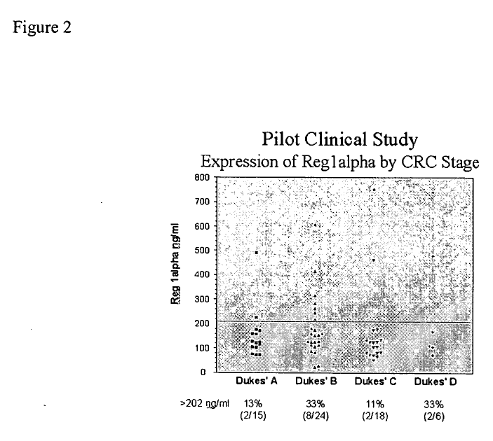

All patients used in this study were diagnosed at their respective medical institutions by qualified physicians using conventional diagnostic means, including physical exam, blood analysis, imaging, and endoscopy. Once identified, patients provided informed consent through an IRB approved protocol. The severity of colorectal cancer in each patient was graded using the Dukes staging scheme. Serum samples were subsequently collected from each patient using methods known to those of skill in the art. Samples were subsequently assessed for the presence of Reg1α by the ELISA assay described above. FIG. 1 shows the levels of Reg1α protein measured in the colorectal cancer patients compared to samples obtained from naïve patients and additional patients diagnosed with either inflammatory bowel disease (IBD) or cirrhosis of the liver. FIG. 2 shows the levels of Reg1α expression in the colorectal cancer patients of FIG. 1, identified at each stage of colorectal cancer severity. As can be seen in FIGS. 1 and 2, Reg1α expression is clearly elevated in serum samples obtained from patients diagnosed with colorectal cancer, and therefore may be used to detect the presence of colorectal cancer in a patient.

### Example 3

### Detection of Reg1α Nucleic Acid Sequence in Colorectal Cancer

In one embodiment, the present invention provides for a method of detecting the presence of colorectal cancer in a patient by detecting the presence of nucleic acid molecules encoding Reg1α in a serum sample obtained from a patient.

Serum may be obtained from a patient suspected of having colorectal cancer by methods described above and known to those of skill in the art. Nucleic acid molecules encoding Reg1α may be detected, for example, by Northern analysis. Briefly, probes for detection of Reg1α mRNA in a patient sample are derived by amplifying the Reg1α coding sequence by RT-PCR according to techniques known in the art. The cDNA fragments generated in this manner are subsequently cloned into a PCRII vector using the TA cloning kit (Invitrogen). The identity of each fragment can be verified by sequencing in each direction from the T3 and T7 polymerase sites present in the cloning vector. The cDNA molecules produced in this manner are then used to produce ³²P-labeled Reg1α cDNA probes using, for example, the Prime-It kit from Stratagene. Subsequently, 5 to 10 μg of total RNA isolated the serum of a patient suspected of having colorectal cancer is separated on an agarose/formaldehyde gel in 1×MOPS buffer. Methods of isolating RNA from a patient sample such as serum are well known in the art (see, for example, Ausubel et al., supra). Following staining with ethidium bromide and visualization under ultra violet light to determine the integrity of the RNA, the RNA is hydrolyzed by treatment with 0.05M NaOH/1.5MNaCl followed by incubation with 0.5M Tris-Cl (pH 7.4)/1.5M NaCl. The RNA is transferred to a commercially available nylon or nitrocellulose membrane (e.g. Hybond-N membrane, Amersham, Arlington Heights, Ill.) by methods well known in the art (Ausubel et al., supra, Sambrook et al., supra). Following transfer and UV cross linking, the membrane is hybridized with a ³²P-labeled Reg1α cDNA probe in hybridization solution (e.g. in 50% formamide/2.5% Denhardt's/100-200 mg denatured salmon sperm DNA/0.1% SDS/5×SSPE) overnight at 65° C. The hybridization conditions can be varied as necessary as described in Ausubel et al., supra and Sambrook et al., supra. Following hybridization, the membrane is washed at room temperature in 2×SSC/0.1% SDS, at 42° C. in 1×SSC/0.1% SDS, at 65° C. in 0.2×SSC/0.1% SDS, and exposed to film overnight with an intensifying screen at −80° C. The stringency of the wash buffers can also be varied depending on the amount of background signal (Ausubel et al., supra). The film is subsequently developed and the intensity bands corresponding to the radiolabeled probe hybridized to RNA are quantified using methods known to those of skill in the art, for example, by digitizing the film and analyzing the band intensity with a computer software program such as NIH Image (NIH, Bethesda, Md.).

Alternatively, Reg1α mRNA may be detected in a patient sample by real-time amplification using oligonucleotide primers capable of specifically hybridizing to the Reg1α a sequence. For example, real-time PCR and TaqMan® probes may be used to detect and quantitate the presence of Reg1α mRNA in a patient sample. The technique of real-time PCR is well known in the art (see, for example, U.S. Pat. Nos. 5,691,146; 5,779,977; 5,866,336; and 5,914,230). Methods of designing primers useful for the amplification of Reg1α sequences are well known in the art (see, for example, Ausubel et al., supra)

cDNA samples, reverse transcribed from mRNA obtained from patient serum samples may be used to generate PCR products via an ABI 7700 sequence detection system (Applied Biosystems, Foster City, Calif.). A measurement may then be made of the level of expression of Reg1α in the patient sample to determine if Reg1α mRNA levels are elevated, thus, providing a means for the detection of colorectal cancer in the patient.

### Example 4

### Detection of Reg1α in Other Patient Samples

In one embodiment of the present invention, colorectal cancer may be detected in a patient by detecting the expression of Reg1α in a clinical patient sample, which is not a serum sample. For example, a circulating cell sample may be obtained from a patient by collecting a sample such as blood, stool, or other bodily fluid. The sample is then subsequently treated to lyse the cells present therein, for example by treating the sample with a suitable lysis buffer, such as a buffer containing 30 mM Tris-Cl, pH 7.4, 100 mM NaCl, 5 mM EDTA, 1% (w/v) SDS, and 100 μg/ml proteinase K (for isolation of nucleic acid). The resulting sample is then analyzed for Reg1α expression either by isolating total RNA from the sample, as described above, and in Ausubel et al., supra, or the sample may be separated on a polyacrylamide gel for analysis by Western blot, or may be utilized in an ELISA-based assay as described above in Example 2.

### Example 5

### Detection of TIMP1 in Patient Serum Samples

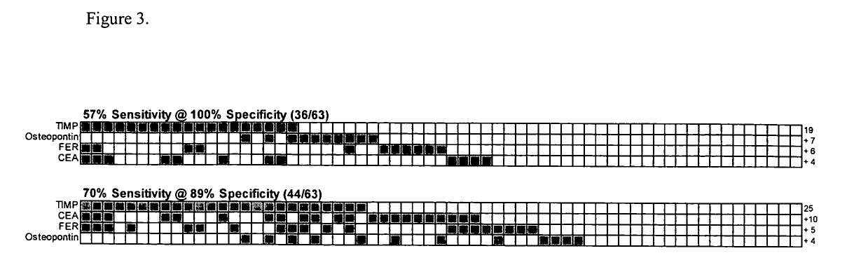

The present invention provides for the detection and monitoring of colorectal cancer in a patient by measuring the level of TIMP1 polypeptide in a patient sample, preferably in a plasma sample. TIMP1 expression was determined in 63 samples from patients diagnosed with colorectal cancer relative to the expression level of TIMP1 in 35 healthy individuals. The results demonstrate that TIMP1, in addition to one or more other colorectal cancer associated markers is overexpressed in colorectal cancer samples relative to normal samples, thus indicating that TIMP1 is a valuable marker for the detection of colorectal cancer in a patient (FIG. 3).

To assess TIMP1 polypeptide expression levels, 63 pre-treatment plasma samples from patients with colorectal cancer, and 35 samples from healthy donors were tested in either commercially available ELISAs (Osteopontin), ADVIA Centaur Immunoassays (CEA and Ferritin), or in-house developed ELISA (TIMP1). All patients used in this study were diagnosed at their respective medical institutions by qualified physicians using conventional diagnostic means, including physical exam, blood analysis, imaging, or endoscopy. Once identified, patients provided informed consent through an IRB approved protocol. The extent of colorectal cancer in each patient was determined using the Dukes' staging scheme. Serum and plasma samples were subsequently collected from each patient using methods known to those of skill in the art.

Specificity at appropriate cutoff values was determined for each marker (e.g., TIMP1, osteopontin, CEA, and ferritin) by evaluating the normal samples. For example, the 100% specificity cutoff for any given marker is equal to the marker value of the highest normal sample. Using these values as the cutoffs, the levels of each marker in the 63 cancer samples were compared to their own respective cutoff values. If the level in the cancer sample was higher than the determined cutoff value, the sample was deemed “positive” and is represented by a shaded box (FIG. 3). This same process was repeated at 97% specificity (using the second highest normal; e.g., 34 of the 35 samples were equal to or below this value). The overall specificity level for the entire panel is calculated by multiplying the specificity of each marker in the panel (e.g., 97%×97%×97%×97%=89% specificity for the panel). The markers were arranged on the graphs shown in FIG. 3, according to the frequency of their overexpression in the cancer samples (TIMP1 was overexpressed in the highest number of cancer patients and is therefore listed first). The marker adding the most to the sensitivity of TIMP1 is ranked second. For example, the 57% sensitivity/100% specificity graph shows that TIMP1 was elevated in 19 of the 63 colorectal cancer patient plasma samples, and is thus listed first on the graph. Evaluating the samples for osteopontin yielded seven additional positive patient samples, and osteopontin is thus listed second on the graph.

The sensitivity of the panel was determined by dividing the cumulative number of samples that were positive for at least one marker by the total number of cancer samples (63).

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A method of diagnosing colon cancer in an individual comprising:
(a) obtaining a serum sample from said individual; and
(b) detecting the presence of TIMP1 in said sample, wherein the presence of Reg1α in said sample is indicative of colon cancer in said individual.

2. The method of claim 1, wherein said step of detecting comprises:
(a) contacting said serum sample with a polypeptide ligand which is capable of binding to TIMP1 under conditions which permit said polypeptide ligand to bind to TIMP1; and
(b) detecting the binding of said polypeptide ligand to TIMP1, wherein detection of binding is indicative of the presence of TIMP1 in said sample.

3. The method of claim 2, wherein said polypeptide ligand is an antibody.

4. The method of claim 2, wherein said polypeptide ligand comprises a detectable label.

5. The method of claim 1, wherein said individual is a human.

6. A method of diagnosing colon cancer in an individual comprising:
(a) obtaining a serum sample from said individual; and
(b) detecting the presence of TIMP1 and at least one other colon cancer specific marker in said sample, wherein the presence of TIMP1 and said at least one other colon cancer-specific marker is indicative of colon cancer in said individual.

7. The method of claim 6, wherein said colon cancer-specific marker is selected from the group consisting of the nucleic acid molecules of SEQ ID Nos 1, 3, 5-71, the polypeptide molecules of SEQ ID Nos 2, 4, 72-138, CA 19-9, CA 72-4, TF, sTn, Tn, CA 50, CA 549, CA 242, LASA, and Du-PAN 1-5.

8. The method of claim 6, wherein said step of detecting comprises:
(a) contacting said serum sample with a first polypeptide ligand which is capable of binding to TIMP1 and a second polypeptide ligand which is capable of binding to said colon cancer-specific marker, under conditions which permit said first and second polypeptide ligands to bind to TIMP1 and said colon cancer-specific marker, respectively; and
(b) detecting the binding of said first polypeptide ligand to TIMP1 and said second polypeptide ligand to said colon cancer-specific marker, wherein detection of binding is indicative of the presence of TIMP1 and said colon cancer-specific marker in said sample.

9. The method of claim 8, wherein said first and second polypeptide ligand are each an antibody.

10. The method of claim 8, wherein said first and second polypeptide ligand comprises a detectable label.

11. The method of claim 6, wherein said individual is a human.

12. A method of diagnosing colon cancer in an individual comprising:
(a) obtaining a serum sample from an individual; and
(b) detecting the presence of a nucleic acid molecule which encodes TIMP1 in said sample, wherein the presence of TIMP1 of said nucleic acid molecule in said sample is indicative of colon cancer in said individual.

13. A method of diagnosing colon cancer in an individual comprising:
(a) obtaining a serum sample from an individual; and
(b) detecting the presence of a nucleic acid molecule which encodes TIMP1 and at least one other nucleic acid molecule which encodes at least one other colon cancer-specific marker in said sample, wherein the presence of said nucleic acid sequence encoding TIMP1 and said nucleic acid sequence encoding said at least one other colon cancer-specific marker is indicative of colon cancer in said individual.

14. The method of claim 13, wherein said colon cancer specific marker is selected from the group consisting of SEQ ID Nos 1, 3, 5-71, the polypeptide molecules of SEQ ID Nos 2, 4, 72-138, CA 19-9, CA 72-4, TF, sTn, Tn, CA 50, CA 549, CA 242, LASA, and Du-PAN 1-5.

15. A method of diagnosing colon cancer in an individual comprising:
(a) obtaining a serum sample from said individual; and
(b) detecting the presence of Reg1α in said sample, wherein the presence of Reg1α in said sample is indicative of colon cancer in said individual.

16. The method of claim 15, wherein said step of detecting comprises:
(a) contacting said serum sample with a polypeptide ligand which is capable of binding to Reg1α under conditions which permit said polypeptide ligand to bind to Reg1α; and
(b) detecting the binding of said polypeptide ligand to Reg1α, wherein detection of binding is indicative of the presence of Reg1α in said sample.

17. The method of claim 16, wherein said polypeptide ligand is an antibody.

18. The method of claim 16, wherein said polypeptide ligand comprises a detectable label.

19. The method of claim 15, wherein said individual is a human.

20. A method of diagnosing colon cancer in an individual comprising:
(a) obtaining a serum sample from said individual; and
(b) detecting the presence of Reg1α and at least one other colon cancer specific marker in said sample, wherein the presence of Reg1α and said at least one other colon cancer-specific marker is indicative of colon cancer in said individual.

21. The method of claim 20, wherein said colon cancer-specific marker is selected from the group consisting of the nucleic acid molecules of SEQ ID Nos 1, 3, 5-71, the polypeptide molecules of SEQ ID Nos 2, 4, 72-138, CA 19-9, CA 72-4, TF, sTn, Tn, CA 50, CA 549, CA 242, LASA, and Du-PAN 1-5.

22. The method of claim 20, wherein said step of detecting comprises:
(a) contacting said serum sample with a first polypeptide ligand which is capable of binding to Reg1α and a second polypeptide ligand which is capable of binding to said colon cancer-specific marker, under conditions which permit said first and second polypeptide ligands to bind to Reg1α and said colon cancer-specific marker, respectively; and
(b) detecting the binding of said first polypeptide ligand to Reg1α and said second polypeptide ligand to said colon cancer-specific marker, wherein detection of binding is indicative of the presence of Reg1α and said colon cancer-specific marker in said sample.

23. The method of claim 22, wherein said first and second polypeptide ligand are each an antibody.

24. The method of claim 22, wherein said first and second polypeptide ligand comprises a detectable label.

25. The method of claim 20, wherein said individual is a human.

26. A method of diagnosing colon cancer in an individual comprising:
(a) obtaining a serum sample from an individual; and
(b) detecting the presence of a nucleic acid molecule which encodes Reg1α in said sample, wherein the presence of Reg1α of said nucleic acid molecule in said sample is indicative of colon cancer in said individual.

27. A method of diagnosing colon cancer in an individual comprising:
(a) obtaining a serum sample from an individual; and
(b) detecting the presence of a nucleic acid molecule which encodes Reg1α and at least one other nucleic acid molecule which encodes at least one other colon cancer-specific marker in said sample, wherein the presence of said nucleic acid sequence encoding Reg1α and said nucleic acid sequence encoding said at least one other colon cancer-specific marker is indicative of colon cancer in said individual.

28. The method of claim 27, wherein said colon cancer specific marker is selected from the group consisting of SEQ ID Nos 1, 3, 5-71, the polypeptide molecules of SEQ ID Nos 2, 4, 72-138, CA 19-9, CA 72-4, TF, sTn, Tn, CA 50, CA 549, CA 242, LASA, and Du-PAN 1-5.
